SEQUENCE LISTING (61 sequences)

SEQ ID NO: 1 - DNA, 20 bp, Artificial Sequence
  Description of Artificial Sequence Oligonucleotide
tccatctccatgaagctact

SEQ ID NO: 2 - DNA, 20 bp, Artificial Sequence
  Description of Artificial Sequence Oligonucleotide
atgaacgaagaccacaaaac

SEQ ID NO: 3 - DNA, 51 bp, Artificial Sequence
  Description of Artificial Sequence Oligonucleotide
ccatctccatgaagctactgcttctgggtaagttttgtggtcttcgttcat

SEQ ID NO: 4 - DNA, 20 bp, Artificial Sequence
  Description of Artificial Sequence Oligonucleotide
gtgtcaagaaggatttgagc

SEQ ID NO: 5 - DNA, 20 bp, Artificial Sequence
  Description of Artificial Sequence Oligonucleotide
tttctgaagcattttggatt

SEQ ID NO: 6 - DNA, 74 bp, Artificial Sequence
  Description of Artificial Sequence Oligonucleotide
gtgtcaagaaggatttgagccggccttatgggagagttaaccggaaacagctcaaatccaaaatgcttcagaaa

SEQ ID NO: 7 - DNA, 20 bp, Artificial Sequence
  Description of Artificial Sequence Oligonucleotide
tctgaagctggactctctgt

SEQ ID NO: 8 - DNA, 20 bp, Artificial Sequence
  Description of Artificial Sequence Oligonucleotide
aatccatagcctcaaactca

SEQ ID NO: 9 - DNA, 100 bp, Artificial Sequence
  Description of Artificial Sequence Oligonucleotide
tctgaagctggactctctgtttgttccattgatccttctcctaagctcatatggcctaacaattatggagtttgggttgatgagtttgaggctatggatt

SEQ ID NO: 10 - DNA, 20 bp, Artificial Sequence
  Description of Artificial Sequence Oligonucleotide
ggctattgttggtggtggtc

SEQ ID NO: 11 - DNA, 20 bp, Artificial Sequence
  Description of Artificial Sequence Oligonucleotide
tccagcttcagaaacctgct

SEQ ID NO: 12 - DNA, 60 bp, Artificial Sequence
  Description of Artificial Sequence Oligonucleotide
gctattgttggtggtggtcctgctggtttagccgtggctcagcaggtttctgaagctgga

SEQ ID NO: 13 - DNA, 20 bp, Artificial Sequence
  Description of Artificial Sequence Oligonucleotide
caaactccactgtggtctgc

SEQ ID NO: 14 - DNA, 20 bp, Artificial Sequence
  Description of Artificial Sequence Oligonucleotide
aacccagtggcatcaagaac

SEQ ID NO: 15 - DNA, 69 bp, Artificial Sequence
  Description of Artificial Sequence Oligonucleotide
aaactccactgtggtctgcagtgacggtgtaaagattcaggcttccgtggttcttgatgccactgggtt

SEQ ID NO: 16 - DNA, 20 bp, Artificial Sequence
  Description of Artificial Sequence Oligonucleotide
tggtgttcatggattggaga

SEQ ID NO: 17 - DNA, 20 bp, Artificial Sequence
  Description of Artificial Sequence Oligonucleotide
gaacgttgggatcttgctgt

SEQ ID NO: 18 - DNA, 80 bp, Artificial Sequence
  Description of Artificial Sequence Oligonucleotide
tggtgttcatggattggagagacaaacatctggactcatatcctgagctgaaagaacggaacagcaagatcccaacgttc

SEQ ID NO: 19 - DNA, 20 bp, Artificial Sequence
  Description of Artificial Sequence Oligonucleotide
ggggatcaatgtgaagagga

SEQ ID NO: 20 - DNA, 20 bp, Artificial Sequence
  Description of Artificial Sequence Oligonucleotide
ccacaacccgttgaggtaag

SEQ ID NO: 21 - DNA, 89 bp, Artificial Sequence
  Description of Artificial Sequence Oligonucleotide
ggggatcaatgtgaagaggattgaggaagacgagcgttgtgtgatcccgatgggcggtcctttaccagtcttacctcaacgggttgtgg

SEQ ID NO: 22 - DNA, 20 bp, Artificial Sequence
  Description of Artificial Sequence Oligonucleotide
gcacccattcaccgagtagt

SEQ ID NO: 23 - DNA, 20 bp, Artificial Sequence
  Description of Artificial Sequence Oligonucleotide
atgttcaacaggtggggaaa

SEQ ID NO: 24 - DNA, 50 bp, Artificial Sequence
  Description of Artificial Sequence Oligonucleotide
gcacccattcaccgagtagtcgaggagacttttccccacctgttgaacat

SEQ ID NO: 25 - DNA, 20 bp, Artificial Sequence
  Description of Artificial Sequence Oligonucleotide
cagtttttgctttgcgttca

SEQ ID NO: 26 - DNA, 20 bp, Artificial Sequence
  Description of Artificial Sequence Oligonucleotide
ctgggcggatttcatctaaa

SEQ ID NO: 27 - DNA, 60 bp, Artificial Sequence
  Description of Artificial Sequence Oligonucleotide
cagtttttgctttgcgttcatttattgaagcctgcaaagatttagatgaaatccgcccag

SEQ ID NO: 28 - DNA, 20 bp, Artificial Sequence
  Description of Artificial Sequence Oligonucleotide
tcaagtgccttctggttgaa

SEQ ID NO: 29 - DNA, 20 bp, Artificial Sequence
  Description of Artificial Sequence Oligonucleotide
agtatgccaagtgccaaagg

SEQ ID NO: 30 - DNA, 70 bp, Artificial Sequence
  Description of Artificial Sequence Oligonucleotide
tcaagtgccttctggttgaagtggttgcaaatgccttttactacaatacccctttggcacttggcatact

SEQ ID NO: 31 - DNA, 20 bp, Artificial Sequence
  Description of Artificial Sequence Oligonucleotide
tcgacactgacaacggtgat

SEQ ID NO: 32 - DNA, 20 bp, Artificial Sequence
  Description of Artificial Sequence Oligonucleotide
ggtactgatggcacggagac

SEQ ID NO: 33 - DNA, 80 bp, Artificial Sequence
  Description of Artificial Sequence Oligonucleotide
tcgacactgacaacggtgatgatgaaactgatgatgctggtgcattggctgcagtgggatgtctccgtgccatcagtacc

SEQ ID NO: 34 - DNA, 20 bp, Artificial Sequence
  Description of Artificial Sequence Oligonucleotide
cgagtctcgtcgatttcctc

SEQ ID NO: 35 - DNA, 20 bp, Artificial Sequence
  Description of Artificial Sequence Oligonucleotide
ttaaagcgaggctaggcaga

SEQ ID NO: 36 - DNA, 91 bp, Artificial Sequence
  Description of Artificial Sequence Oligonucleotide
cgagtctcgtcgatttcctccgggaggagacttgaaattcgtgactttccgattgtgaattccccgatggatctgcctagcctcgctttaa

SEQ ID NO: 37 - DNA, 20 bp, Artificial Sequence
  Description of Artificial Sequence Oligonucleotide
gtctccgtgccatcagtacc

SEQ ID NO: 38 - DNA, 20 bp, Artificial Sequence
  Description of Artificial Sequence Oligonucleotide
agcattttccgcattattgg

SEQ ID NO: 39 - DNA, 100 bp, Artificial Sequence
  Description of Artificial Sequence Oligonucleotide
gtctccgtgccatcagtaccattcttgaatctatcagtagtctccctcatctttatggtcagattgaaccacagttactgccaataatgcggaaaatgct

SEQ ID NO: 40 - DNA, 20 bp, Artificial Sequence
  Description of Artificial Sequence Oligonucleotide
tgtctctgacgacgaggttg

SEQ ID NO: 41 - DNA, 20 bp, Artificial Sequence
  Description of Artificial Sequence Oligonucleotide
cgtcctcttcagcgtcatct

SEQ ID NO: 42 - DNA, 50 bp, Artificial Sequence
  Description of Artificial Sequence Oligonucleotide
tgtctctgacgacgaggttgtccccgtagaagatgacgctgaagaggacg

SEQ ID NO: 43 - DNA, 20 bp, Artificial Sequence
  Description of Artificial Sequence Oligonucleotide
ggagaacgcaaacgtctgtt

SEQ ID NO: 44 - DNA, 20 bp, Artificial Sequence
  Description of Artificial Sequence Oligonucleotide
aagggtgattgcagcatttc

SEQ ID NO: 45 - DNA, 59 bp, Artificial Sequence
  Description of Artificial Sequence Oligonucleotide
ggagaacgcaaacgtctgttgaacatagcaatgcattgcggaaatgctgcaatcaccct

SEQ ID NO: 46 - DNA, 20 bp, Artificial Sequence
  Description of Artificial Sequence Oligonucleotide
aggaaccctcgattcgatct

SEQ ID NO: 47 - DNA, 20 bp, Artificial Sequence
  Description of Artificial Sequence Oligonucleotide
tcgaagctctagccatcgac

SEQ ID NO: 48 - DNA, 69 bp, Artificial Sequence
  Description of Artificial Sequence Oligonucleotide
aggaccctcgattcgatctctcagacgaaatcaggattcgtagaggcgcgtcgatggctagagcttcga

SEQ ID NO: 49 - DNA, 20 bp, Artificial Sequence
  Description of Artificial Sequence Oligonucleotide
ccctcgattcgatctctcag

SEQ ID NO: 50 - DNA, 19 bp, Artificial Sequence
  Description of Artificial Sequence Oligonucleotide
gaagaaacttcccgcttcg

SEQ ID NO: 51 - DNA, 79 bp, Artificial Sequence
  Description of Artificial Sequence Oligonucleotide
cctcgattcgatctctcagacgaaatcaggattcgtagaggcgcgtcgatggctagagctcgaagcgggaagtttcttc

SEQ ID NO: 52 - DNA, 20 bp, Artificial Sequence
  Description of Artificial Sequence Oligonucleotide
cagcaaacgtgagaaggcta

SEQ ID NO: 53 - DNA, 20 bp, Artificial Sequence
  Description of Artificial Sequence Oligonucleotide
tggaagcattttgggagtct

SEQ ID NO: 54 - DNA, 89 bp, Artificial Sequence
  Description of Artificial Sequence Oligonucleotide
cagcaaacgtgagaaggctagactcaaagaaatgcagaagatgaagaagcagaaaattcagcaaatcttagactcccaaaatgcttcca

SEQ ID NO: 55 - DNA, 19 bp, Artificial Sequence
  Description of Artificial Sequence Oligonucleotide
gccgattttgtcctgtcct

SEQ ID NO: 56 - DNA, 20 bp, Artificial Sequence
  Description of Artificial Sequence Oligonucleotide
atgtcgaatttccctgcaac

SEQ ID NO: 57 - DNA, 100 bp, Artificial Sequence
  Description of Artificial Sequence Oligonucleotide
gccgattttgtcctgtcctgcgtgctgtgaaatttctcggtaatcccgaggaaagaagacatattcgtgaagaactgctagttgcagggaaattcgacat

SEQ ID NO: 58 - DNA, 23 bp, Artificial Sequence
  Description of Artificial Sequence Beta Actin Primer
ggtgtgcacttttattcaactgg

SEQ ID NO: 59 - DNA, 20 bp, Artificial Sequence
  Description of Artificial Sequence Beta Actin Primer
agagaagtggggtggctttt

SEQ ID NO: 60 - DNA, 20 bp, Artificial Sequence
  Description of Artificial Sequence Beta Actin Primer
agggcagtgatctccttctg

SEQ ID NO: 61 - DNA, 20 bp, Artificial Sequence
  Description of Artificial Sequence Beta Actin Primer
agaggcgtacagggatagca

(19) United States
(12) Patent Application Publication
(10) Pub. No.: US 2007/0218485 A1
(43) Pub. Date: Sep. 20, 2007

(54) BIOLOGICAL BAR CODE

(75) Inventors: James Davis, Carlsbad, CA (US); Mitchell D. Eggers, Carlsbad, CA (US); Rafael Ibarra, San Diego, CA (US); John Sadler, Belmont, CA (US); David Wong, Escondido, CA (US)

Correspondence Address: PILLSBURY WINTHROP SHAW PITTMAN LLP, ATTENTION: DOCKETING DEPARTMENT, P.O BOX 10500, McLean, VA, 22102, US

(73) Assignee: Gen Vault Corporation, Carlsbad, CA (US)

(21) Appl. No.: 11/678,402

(22) Filed: Feb. 23, 2007

Related U.S. Application Data
(63) Continuation of application No. 10/426,940, filed on Apr. 29, 2003, now abandoned.

(51) Int. Cl. C12Q 1/68; C07H 21/00

(52) U.S. Cl. 435/6; 536/22.1

(57) ABSTRACT

The invention provides compositions and methods useful for identifying, verifying or authenticating any type of sample, whether the sample, is biological or non-biological.

### TECHNICAL FIELD

The invention relates to compositions and methods of identifying samples to ensure their validity, authenticity or accuracy, and more particularly to bar-coded samples and archives, methods of bar-coding samples, and methods of identifying, validating, and authenticating bar-coded samples in which the coding may be done with biological molecules, modified forms or derivatives thereof.

### BACKGROUND

Identification of anonymized DNA samples from human patients can be difficult if the samples are in liquid form and are subject to error during handling. Many other biological and non-biological samples can be confused or subject to identification error. Barcode labels on tubes or containers offer only partial solution of the identification problem as they can fall off, be obscured, removed or otherwise made unreadable. Furthermore, such barcode labels are easily counterfeited. A nucleic acid sample offers a built in identification code but is only useful if the identity information for that nucleic acid is at hand or can be obtained. Long, unique, oligonucleotide sequences have been added to samples as a means of identification but this requires that a unique sequence be synthesized for each and every sample and costly sequencing analysis to identify the oligonucleotide sequences. The invention addresses the inadequacies of present identification methods and provides related advantages.

### SUMMARY

The invention provides compositions allowing identification of a sample, samples uniquely identified by the compositions and methods of producing identified samples and identifying samples so produced. For example, a composition of the invention including two or more oligonucleotides can be added to a sample, in which each of the oligonucleotides do not specifically hybridize to the sample, in which each of the oligonucleotides are physically or chemically different from each other (e.g., their length or sequence), and are in a unique combination that allows identification of the sample.

In one embodiment, a composition includes two or more oligonucleotides and a sample, the oligonucleotides denoted a first oligonucleotide set, the first oligonucleotide set comprising oligonucleotides incapable of specifically hybridizing to said sample, the oligonucleotides having a length from about 8 nucleotides to 50 Kb. The first oligonucleotide set includes oligonucleotides each having a physical or chemical difference from the other oligonucleotides of the first oligonucleotide set, and, optionally the first oligonucleotide set includes one or more oligonucleotides having a different sequence therein capable of specifically hybridizing to a unique primer pair denoted a first primer set. In one aspect, the difference is oligonucleotide length. In various additional aspects, the set includes two oligonucleotides denoted A through B and the unique combination comprises A with or without B; or B with or without A; the set includes three oligonucleotides denoted A through C and the unique combination comprises A with or without B or C; B with or without A or C; or C with or without A or B; the set includes four oligonucleotides denoted A through D and the unique combination comprises A with or without B or C or D; B with or without A or C or D; C with or without A or B or D; or D with or without A or B or C; the set includes five oligonucleotides denoted A through E and the unique combination comprises A with or without B or C or D or E; B with or without A or C or D or E; C with or without A or B or D or E; D with or without A or B or C or E; or E with or without A or B or C or D; the set includes six oligonucleotides denoted A through F and the unique combination comprises A with or without B or C or D or E or F; B with or without A or C or D or E or F; C with or without A or B or D or E or F; D with or without A or B or C or E or F; E with or without A or B or C or D or F; or F with or without A or B or C or D or E; or the set includes seven oligonucleotides denoted A through G and the unique combination comprises A with or without B or C or D or E or F or G; B with or without A or C or D or E or F or G; C with or without A or B or D or E or F or G; D with or without A or B or C or E or F or G; E with or without A or B or C or D or F or G; F with or without A or B or C or D or E or G; or G with or without A or B or C or D or E or F.

In additional embodiments, a unique combination includes two to five, five to ten, 10 to 15, 15 to 20, 20 to 25, 25 to 30, 30 to 40, 40 to 50, 50 to 75, 75 to 100, or more oligonucleotides. Oligonucleotides within a set can have the same or a different sequence length, e.g., differ by at least one nucleotide. In one aspect, the oligonucleotides have a length from about 10 to 5000 base pairs; 10 to 3000 base pairs; 12 to 1000 base pairs; 12 to 500 base pairs; 15 to 250 base pairs; or 18 to 250, 20 to 200, 20 to 150, 25 to 150, 25 to 100, or 25 to 75 base pairs. Oligonucleotides can be single, double or triple strand deoxyribonucleic acid (DNA) or ribonucleic acid (RNA).

In an additional embodiment, a composition includes two or more oligonucleotides and a sample, the two or more oligonucleotides of two or more oligonucleotide sets. In one aspect, a composition therefore includes one or more oligonucleotides denoted a second oligonucleotide set, the second oligonucleotide set including oligonucleotides incapable of specifically hybridizing to the sample, the second oligonucleotide set comprising oligonucleotides having a length from about 8 nucleotides to 50 Kb. The second oligonucleotide set includes oligonucleotides each having a physical or chemical difference from the other oligonucleotides of the second oligonucleotide set, and optionally the second oligonucleotide set includes one or more oligonucleotides having a different sequence therein capable of specifically hybridizing to a unique primer pair denoted a second primer set. In additional aspects, one or more oligonucleotides from additional sets are added to the sample and the one or more oligonucleotides of the first and second oligonucleotide sets, e.g., one or more oligonucleotides denoted a third oligonucleotide set, the third oligonucleotide set including oligonucleotides incapable of specifically hybridizing to the sample, the third oligonucleotide set including oligonucleotides having a length from about 8 nucleotides to 50 Kb, the third oligonucleotide set including oligonucleotides each having a physical or chemical difference from the other oligonucleotides of the third oligonucleotide set and optionally the third oligonucleotide set includes one or more oligonucleotides having a different sequence therein capable of specifically hybridizing to a unique primer pair denoted a third primer set; one or more oligonucleotides denoted a fourth oligonucleotide set, the fourth oligonucleotide set including oligonucleotides incapable of specifically hybridizing to the sample, the fourth oligonucleotide set including oligonucleotides having a length from about 8 nucleotides to 50 Kb, the fourth oligonucleotide set including oligonucleotides each having a physical or chemical difference from the other oligonucleotides of the fourth oligonucleotide set, and optionally the fourth oligonucleotide set includes one or more oligonucleotides having a different sequence therein capable of specifically hybridizing to a unique primer pair denoted a fourth primer set; one or more oligonucleotides denoted a fifth oligonucleotide set, the fifth oligonucleotide set including oligonucleotides incapable of specifically hybridizing to the sample, the fifth oligonucleotide set including oligonucleotides having a length from about 8 nucleotides to 50 Kb, the fifth oligonucleotide set including oligonucleotides each having a physical or chemical difference from the other oligonucleotides of the fifth oligonucleotide set, and optionally the fifth oligonucleotide set includes one or more oligonucleotides having a different sequence therein capable of specifically hybridizing to a unique primer pair denoted a fifth primer set; one or more oligonucleotides denoted a sixth oligonucleotide set, the sixth oligonucleotide set including oligonucleotides incapable of specifically hybridizing to the sample, the sixth oligonucleotide set including oligonucleotides having a length from about 8 nucleotides to 50 Kb, the sixth oligonucleotide set including oligonucleotides each having a physical or chemical difference from the other oligonucleotides of the sixth oligonucleotide set and optionally the sixth oligonucleotide set includes one or more oligonucleotides having a different sequence therein capable of specifically hybridizing to a unique primer pair denoted a sixth primer set; and so on and so forth. In a particular aspect, the difference is in oligonucleotide length. In additional aspects, the one or more oligonucleotides of the first, second, third, fourth, fifth, sixth, etc., oligonucleotide set has the same or a different length as an oligonucleotide of the first, second, third, fourth, fifth, sixth, etc., oligonucleotide set. In further aspects, the one or more oligonucleotides of each additional oligonucleotide set, e.g., third, fourth, fifth, sixth, etc., has the same or a different length as an oligonucleotide of the first, second, third, fourth, etc. oligonucleotide set. Thus, for example, in one aspect, an oligonucleotide of the first, second, third, fourth, fifth or sixth oligonucleotide set has the same or a different length as an oligonucleotide of the second, third, fourth or fifth oligonucleotide set, respectively.

In yet additional embodiments, a composition includes one or more unique primer pairs of a primer set, e.g., a composition that includes oligonucleotides denoted a first, second, third, fourth, fifthi, sixth, etc., set includes a first primer set that specifically hybridizes to one or more of the oligonucleotides denoted the first set. In still further embodiments, a composition that includes oligonucleotides denoted a first, second, third, fourth, fifth, or sixth, etc., set includes a first, second, third, fourth, fifth, or sixth, etc. primer set that specifically hybridizes to one or more of the oligonucleotides denoted the first, second, third, fourth, fifth, or sixth, etc. set. The primers of the unique primer pairs can have any length, e.g., a length from about 8 to 250, 10 to 200, 10 to 150, 10 to 125, 12 to 100, 12 to 75, 15 to 60, 15 to 50, 18 to 50, 20 to 40, 25 to 40 or 25 to 35 nucleotides. The primers of the unique primer pairs can have a length of about 9/10, 4/5, 3/4, 7/10, 3/5, 1/2, 2/5, 1/3, 3/10, 1/4, 1/5, 1/6, 1/7, 1/8, 1/10 of the length of the oligonucleotide to which the primer binds. Primers can bind at or near the 3′ or 5′ terminus of the oligonucleotide, e.g., within about 1 to 25 nucleotides of the 3′ or 5′ terminus of the oligonucleotide. Primers can have the same or different lengths, e.g., each primer of the unique primer pair differs in length from about 0 to 50, 0 to 25, 0 to 10, or 0 to 5 base pairs; can be entirely or partially complementary to all or at least a part of one or more of the oligonucleotides, e.g., 40-60%, 60-80%, 80-95% or more (primers need not be 100% homologous or have 100% complementarity); and can be 100% complementary to a sequence.

Samples include any physical entity. Exemplary samples include pharmaceuticals, biologicals and non-biological samples. Non-biological samples include any document (e.g., evidentiary document, a testamentary document, an identification card, a birth certificate, a signature card, a driver's license, a social security card, a green card, a passport, a letter, or a credit or debit card), currency, bond, stock certificate, contract, label, piece of art, recording medium (e.g., digital recording medium), electronic device, mechanical or musical instrument, precious stone or metal, or dangerous device (e.g., firearm, ammunition, an explosive or a composition suitable for preparing an explosive).

Biological samples include foods (meats or vegetables such as beef, pork, lamb, fowl or fish), beverages (alcohol or non-alcohol). Biological samples include tissue samples, forensic samples, and

fluids such as blood, plasma, serum, sputum, semen, urine, mucus, cerebrospinal fluid and

stool. Biological samples further include any living or non-living cell, such as an egg or sperm,

bacteria or virus, pathogen, nucleic acid (mammalian such as human or non-mammalian), protein, carbohydrate. Typically, a sample that is nucleic acid will have less than 50% homology with the different sequence of the oligonucleotides or the primer pairs, such that the oligonucleotides or primer pairs do not specifically hybridize to the human nucleic acid to the extent that it prevents developing the code. Thus, in particular aspects, for a nucleic acid that is bacterial the oligonucleotides do not specifically hybridize to the bacterial nucleic acid, for a nucleic acid that is viral the oligonucleotides do not specifically hybridize to the viral nucleic acid.

Oligonucleotides can be modified, e.g., to be nuclease resistant. Compositions can include

preservatives, e.g., nuclease inhibitors such as EDTA, EGTA, guanidine thiocyanate or uric acid. Oligonucleotides can be mixed with, added to or imbedded within the sample, e.g., attached to, applied to, affixed to or imbedded within a substrate (permeable, semi-permeable or impermeable two dimensional surface or three dimensional structure, e g., a plurality of wells). Oligonucleotides can be

physically separable or inseparable from the substrate, e.g., under conditions where the sample remains substantially attached to the substrate the oligonucleotides can be separated.

In yet further embodiments, a composition includes three or more unique primer pairs and two or more oligonucleotides, optionally in combination with a sample, wherein the unique primer pairs are denoted a first, second, third, fourth, fifth, or sixth, etc. primer set, each of the unique primer pairs having a different sequence, at least two of the unique primer pairs capable of specifically hybridizing to two oligonucleotides, wherein the oligonucleotides are denoted a first, second, third, fourth, fifth, or sixth, etc. oligonucleotide set, the oligonucleotides having a length from about 8 nucleotides to 50 Kb. The oligonucleotides in each set have a physical or chemical difference from the other oligonucleotides comprising the same oligonucleotide set. In various aspects, a composition includes additional unique primer pairs, e.g., four or more unique primer pairs, five or more unique primer pairs, six or more unique primer pairs. In additional aspects, a composition includes additional oligonucleotides, e.g.,

three, four, five, six or more oligonucleotides, etc. In still further aspects, a composition includes one or more oligonucleotides denoted a second, third, fourth, fifth, sixth, etc. oligonucleotide set, the oligonucleotide(s) of the second, third, fourth, fifth, sixth, etc. oligonucleotide set including one or more oligonucleotides having a different sequence therein capable of specifically hybridizing to a unique corresponding primer pair denoted a second, third, fourth, fifth, sixth, etc. primer set, the second, third, fourth, fifth, sixth, etc. oligonucleotide set including oligonucleotides incapable of specifically hybridizing to the sample, the second, third, fourth, fifth, sixth, etc. oligonucleotide set including oligonucleotides having a length from about 8 nucleotides to 50 Kb, the second, third, fourth, fifth, sixth, etc. oligonucleotide set including oligonucleotides each having a physical or chemical difference from the other oligonucleotides comprising the second, third, fourth, fifth, sixth, etc. oligonucleotide set.

In still additional embodiments, a composition of the invention is in an organic or aqueous solution having one or more phases (compatible with polymerase chain reaction (PCR)), slurry, semi-solid, or a solid. In further embodiments, a composition of the invention is included within a kit.

The invention also provides methods of producing bio-tagged samples. In one embodiment, a method includes selecting a combination of two or more oligonucleotides to add to a sample, the oligonucleotides, optionally from two or more oligonucleotide sets, incapable of specifically hybridizing to the sample, the oligonucleotides having a length from about 8 to 5000 nucleotides, and the oligonucleotides within each set having a physical or chemical difference (e.g., oligonucleotide length), and adding the combination of two or more oligonucleotides to the sample, wherein the combination of oligonucleotides identifies the sample, thereby producing a bio-tagged sample. In one aspect, one or more of the oligonucleotides has a different sequence therein capable of specifically hybridizing to a unique primer pair.

The invention further provides methods of identifying bio-tagged samples. In one embodiment, a method includes detecting in a sample the presence or absence of two or more oligonucleotides, wherein the oligonucleotides are identified based upon a physical or chemical difference, thereby identifying a combination of oligonucleotides in the sample; comparing the combination of oligonucleotides with a database including particular oligonucleotide combinations known to identify particular samples; and identifying the sample based upon which of the particular oligonucleotide combinations in the database is identical to the combination of oligonucleotides in the sample. In one aspect, sample identification is based upon the different lengths of the oligonucleotides. In another aspect, sample identification is based upon the different sequence of the oligonucleotides. In yet another aspect, identification does not require sequencing all of the oligonucleotides, e.g., identification is based upon a primer or primer pairs that specifically hybridizes to one or more of the oligonucleotides that identifies the sample. In still another aspect, identification is based upon the different lengths of the oligonucleotides, or by hybridization to two or more unique primer pairs having a different sequence, optionally followed by amplification (e.g., PCR). The method of claim 118, wherein the oligonucleotides are selected.

The invention moreover provides archives of bio-tagged samples. In one embodiment, an archive includes a sample; and two or more oligonucleotides. The oligonucleotides are incapable of specifically hybridizing to the sample, the oligonucleotides have a length from about 8 to 50 Kb nucleotides, the oligonucleotides each have a physical or chemical difference (e.g., a different length), and optionally one or more of the oligonucleotides have a different sequence therein capable of specifically hybridizing to a unique primer pair, the oligonucleotides are in a unique combination that identifies the sample; and a storage medium for storing the bio-tagged samples.

The invention still further provides methods of producing archives of bio-tagged samples. In one embodiment, a method includes selecting a combination of two or more oligonucleotides to add to a sample, the oligonucleotides are incapable of specifically hybridizing to the sample, the oligonucleotides have a length from about 8 to 50 Kb nucleotides, the oligonucleotides each have a physical or chemical difference (e.g., a different length), one or more of the oligonucleotides have a different sequence therein capable of specifically hybridizing to a unique primer pair; adding the combination of two or more oligonucleotides to the sample and placing the bio-tagged sample in a storage medium for storing the bio-tagged samples. The combination of oligonucleotides identifies the sample.

### DESCRIPTION OF DRAWINGS

FIGS. 1A and 1B illustrate exemplary codes, A) 534523151, or in binary form, 10100 01000 10010 00101 10001 and B) 530523151, or in binary form, 10100 00000 10010 00101 10001, following size-based fractionation of amplified oligonucleotides. Lanes are as follows: 1, a ladder of 5 oligonucleotides with lengths of 60, 70, 80, 90, and 100 nucleotides; 2, primer set #1 amplified oligonucleotides; 3, primer set #2 amplified oligonucleotides; 4, primer set #3 amplified oligonucleotides; 5, primer set #4 amplified oligonucleotides; 6, primer set #5 amplified oligonucleotides. Sets 1-5 are multiplex primer sets for each of the 5 oligonucleotide sets.

### DETAILED DESCRIPTION

The invention is based at least in part on compositions including oligonucleotides that are physically or chemically different from each other (e.g., in their length and/or sequence), and that are in a unique combination. Adding to or mixing a unique combination of oligonucleotides with a given sample, i.e., coding the sample, allows the sample to be identified based upon the combination of oligonucleotides added or mixed. By determining the oligonucleotide combination (the “code”) in a query sample and comparing the oligonucleotide combination to oligonucleotide combinations known to identify particular samples (e.g., a database of known oligonucleotide combinations that identify samples), the query sample is thereby identified. Thus, where it is desired to identify, verify or authenticate a sample, a unique combination of oligonucleotides can be added to or mixed with the sample, and the sample can subsequently be identified, verified or authenticated based upon the particular unique combination of oligonucleotides present in the sample.

As a non-limiting illustration of the invention, from a pool of 25 oligonucleotides, each oligonucleotide having a different sequence and each oligonucleotide having a different length (in this example, five lengths: 60, 70, 80, 90 and 100 nucleotides), nine are added to a sample. The nine oligonucleotides added to the sample (the “code”) are recorded and the code optionally stored in a database. The oligonucleotide code is developed using primer pairs that specifically hybridize to each oligonucleotide that is present. In this particular illustration, there are 25 oligonucleotides possible and 5 sets of primer pairs (denoted primer Sets 1-5). Each set of primer pairs specifically hybridize to 5 oligonucleotides and, therefore, by using 5 primer sets, all 25 oligonucleotides potentially present in the sample are identified. In this illustration, the nine oligonucleotides present in the sample which specifically hybridize to a corresponding primer pair are identified by polymerase chain reaction (PCR) based amplification. In contrast, because the other 16 oligonucleotides are absent from the sample these oligonucleotides will not be amplified by the primers that specifically hybridize to them. Thus, differential primer hybridization among the different oligonucleotides is used to identify which oligonucleotides, among those possibly present, that are actually present in the sample.

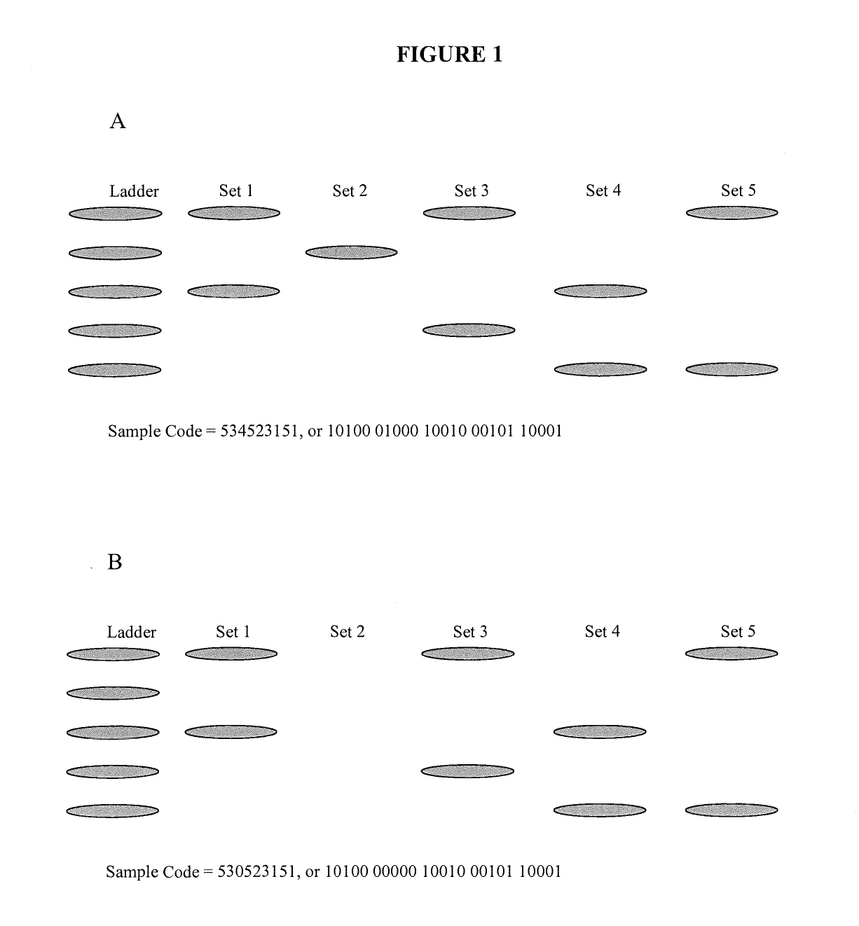

Following PCR, the 5 reactions containing amplified products, which in this illustration reflect both the oligonucleotide length and the sequence of the region that hybridizes to the primers, are size-fractionated via gel electrophoresis: each reaction representing one primer set is fractionated in a single lane for a total of 5 lanes (Sets 1-5, which correspond to FIG. 1, lanes 2-6, respectively). The developed “bar-code” in this illustration is the pattern of the fractionated amplified products in each lane. In this illustration, the 60, 70, 80, 90 and 100 base oligonucleotides correspond to code numbers 1, 2, 3, 4 and 5, respectively, and the bar code is read beginning with lane 2, from top to bottom, and each lane thereafter, 534523151 (FIG. 1A). Alternatively, the bar-code may be designated as a binary number, where each of the 25 possible oligonucleotides at the 60, 70, 80, 90 and 100 positions in all 5 lanes is designated by a “1” or a “0” based upon the presence or absence, respectively, of the oligonucleotide (amplified product) at that particular position. Thus, in FIG. 1A the corresponding binary number would read 10100 01000 10010 00101 100001.

In the exemplary illustration each primer set amplifies at least one oligonucleotide. However, because not all oligonucleotides need be present, oligonucleotides for a given primer set may be completely absent. That is, a code where an oligonucleotide is absent is designated by a “0.” Thus, for example, where there is no oligonucleotide present that specifically hybridizes to a primer pair in primer set #2, the code would read: 530523151 (FIG. 1B), and the corresponding binary number for lane 2 would be “0” at each position, which would read 10100 00000 10010 00101 10001.

In order to develop the “code” in the exemplary illustration, every primer pair that specifically hybridizes to every oligonucleotide from the pool of 25 oligonucleotides is used in the amplification reactions. The initial screen for which oligonucleotides are actually present in the sample is therefore based upon differential primer hybridization and subsequent amplification of the oligonucleotide(s) that hybridizes to a corresponding primer pair. Thus, every one of the 25 oligonucleotides potentially present in the sample can be identified because all primer pairs that specifically hybridizes to all oligonucleotides are used in the screen. In the illustration, five primer sets are used, each primer set containing 5 primer pairs. Five separate reactions were performed with the 5 primer pairs in each primer set to amplify all 25 oligonucleotides. Thus, although primer pair may be present in any given reaction, if the oligonucleotide that specifically hybridizes to the primer pair is absent from that reaction, the oligonucleotide will not be amplified.

Following the reactions, the oligonucleotides (amplified products) are differentiated from each other based upon differences in their length. Thus, in the context of developing the code, oligonucleotides comprising the code need not be subject to sequencing analysis in order to identify or distinguish them from one another. Accordingly, the invention does not require that the oligonucleotides comprising the code be sequenced in order to develop the code.

In the exemplary illustration, the “code” is developed by dividing the sample containing the oligonucleotides into five reactions and separately amplifying the reactions with each primer set. For example, a coded sample that is applied or attached to a substrate (e.g., a small 3 mm diameter matrix) can be divided into 5 pieces and the amplification reactions performed on each the 5 pieces of substrate, each reaction having a different primer set. Optionally, the oligonucleotides could first be eluted from the substrate and the eluent divided into five separate reactions. As an alternative approach to separate reactions, the substrate can be subjected to 5 sequential reactions with each primer set. For example, if the oligonucleotide code is applied or attached to a substrate the code can be developed by performing 5 sequential amplification reactions on the substrate, and removing the amplified products after each reaction before proceeding to the next reaction. The amplified products from each of the 5 reactions are then fractionated separately to develop the code.

If desired fewer oligonucleotides can be used, optionally in a single dimension. A set of oligonucleotides or amplified products can be fractionated in a single dimension, e.g., one lane. For example, where a large number of unique codes is not anticipated to be needed 2, 3, 4, 5, 6, 7, 8, 9, 10, etc. oligonucleotides can be a code in a single lane format. A corresponding single primer set would therefore include 2, 3, 4, 5, 6, 7, 8, 9, 10, etc. numbers of unique primer pairs in order to detect/identify the 2, 3, 4, 5, 6, 7, 8, 9, 10, oligonucleotides, respectively, that may be present. Given sufficient resolving power of the separation system, essentially there is no upper limit to the number of oligonucleotides that can be separated in one dimension. Thus, there may be 10-15, 15-20, 20-25, 25-30, 30-35, 35-40, 40-45, 45-50, etc., or more oligonucleotides that may be separated in a single dimension. Accordingly, invention compositions can contain unlimited numbers of oligonucleotides in one or more oligonucleotide sets. A given primer set therefore also need not be limited; the number of primer pairs in a primer set will reflect the number of oligonucleotides desired to be amplified, e.g., 10-15, 15-20, 20-25, 25-30, 30-35, 35-40, 40-45, 45-50, etc., or more oligonucleotides.

Thus, in one embodiment the invention provides compositions including two or more oligonucleotides and a sample; the oligonucleotides denoted a first oligonucleotide set, the first oligonucleotide set including oligonucleotides incapable of specifically hybridizing to the sample, the first oligonucleotide set oligonucleotides having a length from about 8 to 50 Kb nucleotides, the first oligonucleotide set oligonucleotides each having a physical or chemical difference (e.g., a different length) from the other oligonucleotides comprising the first oligonucleotide set, and the first oligonucleotide set oligonucleotides each having a different sequence therein capable of specifically hybridizing to a unique primer pair denoted a first primer set. In one aspect, the first oligonucleotide set oligonucleotides are in a unique combination allowing identification of the sample. In additional aspects, the two oligonucleotides are denoted A and B, and the composition includes A with or without B, or B alone; the three oligonucleotides are denoted A through C and the composition includes A with or without B or C, B with or without A or C, or C with or without A or B; the four oligonucleotides are denoted A through D and the composition includes A with or without B or C or D, B with or without A or C or D, C with or without A or B or D, or D with or without A or B or C; the five oligonucleotides are denoted A through E and the compositions includes A with or without B or C or D or E, B with or without A or C or D or E, C with or without A or B or D or E, D with or without A or B or C or E, or E with or without A or B or C or D; the six oligonucleotides are denoted A through F and the composition includes A with or without B or C or D or E or F, B with or without A or C or D or E or F, C with or without A or B or D or E or F, D with or without A or B or C or E or F, E with or without A or B or C or D or F, or F with or without A or B or C or D or E; the seven oligonucleotides are denoted A through G and the composition includes A with or without B or C or D or E or F or G, B with or without A or C or D or E or F or G, C with or without A or B or D or E or F or G, D with or without A or B or C or E or F or G, E with or without A or B or C or D or F or G, F with or without A or B or C or D or E or G, or G with or without A or B or C or D or E or F. In yet further aspects, the first oligonucleotide set includes a unique combination of two to five, five to ten, 10 to 15, 15 to 20, 20 to 25, 25 to 30, 30 to 40, 40 to 50, 50 to 100, or more oligonucleotides.

As used herein, the term “physical or chemical difference,” and grammatical variations thereof, when used in reference to oligonucleotide(s), means that the oligonucleotide(s) has a physical or chemical characteristic that allows one or more of the oligonucleotides to be distinguished from each another. In other words, the oligonucleotides have a difference that allows them to be distinguished from one or more other oligonucleotides and, therefore, identified when present among the other oligonucleotides. One particular example of a physical difference is oligonucleotide length. Another particular example of a physical difference is oligonucleotide sequence. Additional examples of physical differences that allow oligonucleotides to be distinguished from each other, which may in part be influenced by oligonucleotide length or sequence, include charge, solubility, diffusion rate, and absorption. Examples of chemical differences include modifications as set forth herein, such as molecular beacons, radioisotopes, fluorescent moieties, and other labels. As discussed, when developing the code sequencing of the oligonucleotides is not required.

Generally, as used herein for convenience purposes the oligonucleotide sets are designated according to the primer sets used to amplify them. Thus, in the exemplary illustration, primer set #1 amplifies oligonucleotide set #1; primer set #2 amplifies oligonucleotide set #2; primer set #3 amplifies oligonucleotide set #3; primer set #4 amplifies oligonucleotide set #4; primer set #5 amplifies oligonucleotide set #5; primer set #6 amplifies oligonucleotide set #6; primer set #7 amplifies oligonucleotide set #7; primer set #8 amplifies oligonucleotide set #8, primer set #9 amplifies oligonucleotide set #9; primer set #10 amplifies oligonucleotide set #10, etc.

In the above exemplary illustration, primer set #1 amplified products (oligonucleotides) are size-fractionated in lane 2, primer set #2 amplified products (oligonucleotides) are size-fractionated in lane 3, primer set#3 amplified products (oligonucleotides) are size-fractionated in lane 4, primer set#4 amplified products (oligonucleotides) are size-fractionated in lane 5, and primer set#5 amplified products (oligonucleotides) are size-fractionated in lane 6 (FIG. 1). However, amplified products need not be fractionated in any particular lane in order to obtain the correct code, provided that the primers used to produce the amplified products are known and the reactions are separately fractionated. That is, by knowing which primers are used in the amplification reaction, e.g., primer set #1 specifically hybridizes to and amplifies oligonucleotides of set #1, the amplified products and, therefore, the oligonucleotides detectable are also known. Thus, amplified products can be fractionated in any order (lane) since the primers that specifically hybridize to particular oligonucleotides are known. For example, if the correct code is obtained by reading the amplified products from primer sets #1-#5 in order, but the primer sets are fractionated out of order, (e.g., primer set #1 is run in lane 2 and primer set #2 is run in lane 1) the code can be corrected by merely reading lane 2 (primer set #1) before lane 1 (primer set #2). Accordingly, amplified products can be fractionated in any order to develop the code because they can be “read” to correspond with the order of the primer set that provides the correct code.

In the exemplary illustration, oligonucleotides amplified with primer sets #1-5 are separately size fractionated in 5 lanes to develop the code (FIG. 1, five lanes, beginning with primer set #1 in lane 2). Even though an invention code can be employed in which oligonucleotides are fractionated in a single lane following amplification with one primer set, using multiple primer sets and fractionating oligonucleotides in multiple lanes provides a more convenient format and expands the number of unique codes available within that format in comparison to fractionating in a single dimension (one lane). The number of different code combinations can be represented as 2^{n(m)}, where “n” represents the number of oligonucleotides per lane and “m” represents the number of lanes. Thus, in the exemplary illustration, 25 oligonucleotides in a 5×5 format (5 oligonucleotides per lane in 5 lanes) provides 2²⁵ different code combinations, or 33,554,432 codes. In contrast, 5 oligonucleotides in a 5×1 format (5 oligonucleotides in one lane) provides 2⁵ different code combinations, or 32 codes

In the exemplary illustration the amplified products fractionated in a single lane (one set of oligonucleotides corresponding to one primer set) are physically or chemically different from each other (e.g., have a different length, charge, solubility, diffusion rate, adsorption, or label) in order to be distinguished from each other. Thus, in addition to increasing the number of available codes, an advantage of fractionating in multiple lanes is that the oligonucleotides or amplified products fractionated in different lanes can have one or more identical physical or chemical characteristics yet still be distinguished from each other. For example, using two dimensions allows oligonucleotides in different sets to have the same length since each set is separately fractionated from the other set(s) (e.g., each set is fractionated in a different lane). Furthermore, each oligonucleotide can have the same sequence. As the number of oligonucleotides fractionated in a given lane increase, a broader size range for the oligonucleotides in order to fractionate them and, consequently, greater resolving power of the fractionation system may be needed in order to develop the code. Thus, where length is used to distinguish between the oligonucleotides within a given set, because the oligonucleotides in different sets can have identical lengths, the oligonucleotides used for the code can have a narrower size range and be fractionated with comparatively less resolving power. The use of multiple dimensions for size fractionation is also more convenient than one dimension since fewer primers are present in a given reaction mix.

Thus, in accordance with the invention there are provided compositions including multiple oligonucleotide sets and a sample. In one embodiment, oligonucleotides denoted a first oligonucleotide set include oligonucleotides incapable of specifically hybridizing to the sample, the oligonucleotides having a length from about 8 to 50 Kb nucleotides, oligonucleotides each having a physical or chemical difference (e.g., a different length) from the other oligonucleotides comprising the first oligonucleotide set, the oligonucleotides each having a different sequence therein capable of specifically hybridizing to a unique primer pair denoted a first primer set; and oligonucleotides denoted a second oligonucleotide set include oligonucleotides each having a different sequence therein capable of specifically hybridizing to a unique primer pair denoted a second primer set, incapable of specifically hybridizing to the sample, a length from about 8 to 50 Kb nucleotides, and each have a physical or chemical difference (e.g., a different length) from the other oligonucleotides comprising said second oligonucleotide set.

In another embodiment, compositions include two oligonucleotide sets and a third oligonucleotide set, the third oligonucleotide set including oligonucleotides each having a different sequence therein capable of specifically hybridizing to a unique primer pair denoted a third primer set, incapable of specifically hybridizing to the sample, a length from about 8 to 50 Kb nucleotides, and each having a physical or chemical difference (e.g., a different length) from the other oligonucleotides of the third oligonucleotide set.

In a further embodiment, compositions include three oligonucleotide sets and a fourth oligonucleotide set, the fourth oligonucleotide set including oligonucleotides each having a different sequence therein capable of specifically hybridizing to a unique primer pair denoted a fourth primer set, incapable of specifically hybridizing to the sample, a length from about 8 to 50 Kb nucleotides, and each having physical or chemical difference (e.g., a different length) from the other oligonucleotides of the fourth oligonucleotide set.

In an additional embodiment, compositions include four oligonucleotide sets and a fifth oligonucleotide set, the fifth oligonucleotide set including oligonucleotides each having a different sequence therein capable of specifically hybridizing to a unique primer pair denoted a fifth primer set, incapable of specifically hybridizing to the sample, a length from about 8 to 50 Kb nucleotides, and each having a physical or chemical difference (e.g., a different length) from the other oligonucleotides of the fifth oligonucleotide set. In various aspects of the invention, in the compositions including multiple oligonucleotide sets, one or more oligonucleotides of the second, third, fourth, fifth, sixth, etc., oligonucleotide set has a physical or chemical characteristic that is the same as one or more oligonucleotides of any other oligonucleotide set (e.g., an identical nucleotide length).

The number of oligonucleotides that may be selected from for producing a coded sample may initially be large enough to account for potentially large numbers of samples or be increased as the number of samples coded increases. For example, where there are few samples to be coded, in one dimension (one lane), 2 unique oligonucleotides provide 4 unique codes (2²), e.g., in binary form, 00, 01, 10, 11; for 3 unique oligonucleotides 8 unique codes are available (2³), e.g., in binary form, 000, 001, 010, 100, 011, 110, 101, 111; for 4 unique oligonucleotides 16 unique codes are available (2⁴); for 5 unique oligonucleotides 32 unique codes are available (2⁵). To expand the number of available codes, one need only increase the number of different oligonucleotides. For example, for 6 unique oligonucleotides 64 unique codes are available (2⁶); for 7 unique oligonucleotides 128 unique codes are available (2⁷); for 8 there are 256 codes available; for 9 there are 512 codes available; for 10 there are 1,024 codes available; for 11 there are 2,048 codes available; for 12 there are 4,096 codes available; for 13 there are 8,192 codes available; for 14 there are 16,384 codes available; for 15 there are 32,768 codes available; for 16 there are 65,536 codes available; for 17 there are 131,072 codes available; for 18 there are 262,144 codes available; for 19 there are 524,288 codes available; for 20 there are 1,048,576 codes available; for 21 there are 2,097,152 codes available; for 22 there are 4,194,304 codes available; for 23 there are 8,388,608 codes available; for 24 there are 16,777,216 codes available; for 25 there are 33,554,432 codes available; etc. Thus, where the number of samples exceeds the available codes, where there are an unknown number of samples to be coded, or where it is desired that the number of codes available be in excess of the projected number samples, additional different oligonucleotides may be added to the oligonucleotide pool from which the oligonucleotides are selected for the code, or the coding may employ an initial large number of different oligonucleotides in order to provide an unlimited number of unique oligonucleotide combinations and, therefore, unique codes. For example, 30 different oligonucleotides provides over one billion unique codes (1,073,741,824 to be precise).

A third dimension could be added in order to expand the code. Adding a third dimension would expand the number of codes available to 2^{(m)np}, where “p” represents the third dimension. Thus, adding a third dimension to a 5×5 format as in the exemplary illustration, 2^{25(p)} different unique codes are available. One example of a third dimension could be based upon isoelectric point or molecular weight. For example, a unique peptide tag could be added to one or more of the oligonucleotides and the code fractionated using isoelectric focusing or molecular weight alone, or in combination, e.g. 2D gel electrophoresis.

The code can include additional information. For example, a code can include a check code. By using the number of oligonucleotides in each lane a check can be embedded with the code. For example, in FIG. 1A, lanes 2-6 have 2, 1, 2, 2 and 2 oligonucleotides, respectively. The check code in this case would be 21222. For FIG. 1B, the check code would be 20222.

The code output can be “hashed,” if desired, so that the code loses any characteristics that would allow it to be traced back to the original sample or the patient that provided the sample. For example, each number in 534523151 could be increased or decreased by one, 645634262 and 423412040, respectively.

The term “hybridization,” “annealing” and grammatical variations thereof refers to the binding between complementary nucleic acid sequences. The term “specific hybridization,” when used in reference to an oligonucleotide capable of forming a non-covalent bond with another sequence (e.g., a primer), or when used in reference to a primer capable of forming a non-covalent bond with another sequence (e.g., an oligonucleotide) means that the hybridization is selective between 1) the oligonucleotide and 2) the primer. In other words, the primer and oligonucleotide preferentially hybridize to each other over other nucleic acid sequences that may be present (e.g., other oligonucleotides, primers, a sample that is nucleic acid, etc.) to the extent that the oligonucleotides present can be identified to develop the code.

Suitable positive and negative controls, for example, target and non-target oligonucleotides or other nucleic acid can be tested for amplification with a particular primer pair to ensure that the primer pair is specific for the target oligonucleotide. Thus, the target oligonucleotide, if present, is amplified by the primer pair whereas the non-target oligonucleotides, non-target primers or other nucleic acid are not amplified to the extent they interfere with developing the code. False negatives, i.e., where an oligonucleotide of the code is present but not detected following amplification, can be detected by correlating the oligonucleotides of the code that are detected with the various codes that are possible. For example, a gel scan of the correct code(s) can be provided to the end user in order to allow the user to match the code detected with one of the gel scan codes. Where the end user is dealing with a limited number of codes, even if one or a few oligonucleotides are not detected, the correct code can readily be identified by matching the detected code with the gel scan of the possible codes that may be available, particularly where the number of available codes possible is large. More particularly for example, an end user requests 10 coded samples from an archive for sample analysis. The coded samples are retrieved from the archive and forwarded to the end user who subsequently analyzes the samples. In order to ensure that a particular sample subsequently analyzed corresponds to the sample received from the archive, the end user then wishes to determine the code for that sample. However, one of the oligonucleotides of the code in that sample is not detected during the analysis of the code, producing an incomplete code. Because the codes for all samples forwarded to the end user are known, the incomplete code can be fully completed based on the code to which the incomplete code most closely corresponds. Alternatively, all codes received by the end user could be developed and, by a process of elimination the incomplete code is developed.

For two nucleic acid sequences to hybridize, the temperature of a hybridization reaction must be less than the calculated TM (melting temperature). As is understood by those skilled in the art, the TM refers to the temperature at which binding between complementary sequences is no longer stable. The TM is influenced by the amount of sequence complementarity, length, composition (% GC), type of nucleic acid (RNA vs. DNA), and the amount of salt, detergent and other components in the reaction. For example, longer hybridizing sequences are stable at higher temperatures. Duplex stability between RNAs or DNAs is generally in the order of RNA:RNA>RNA:DNA>DNA:DNA. All of these factors are considered in establishing appropriate conditions to achieve specific hybridization (see, e.g., the hybridization techniques and formula for calculating TM described in Sambrook et al., 1989, supra). Generally, stringent conditions are selected to be about 5° C. lower than the melting point (Tm) for the specific sequence at a defined ionic strength and pH.

Exemplary conditions used for specific hybridization and subsequent amplification for developing the exemplary code are disclosed in Example 1. One exemplary condition for PCR is as follows: Buffer (1X): 16 mM (NH₄)₂SO₄, 67 mM Tris-HCl (pH 8.8 at 25 C), 0.01% Tween 20, 1.5 mM MgCl₂; dNTP: 200 uM each; Primer concentration: 62.5 mM of each primer (all 5 primer pairs present in each reaction); Enzyme: 2 units of Biolase (Taq; Bioline, Randolph, M A); PCR cycling conditions: 93 C for 2 minutes, 55 C for 1 minute, 72 C for 2 minutes, followed by 29 cycles of 93 C for 30 seconds, 55 C for 30 seconds, 72 C for 45 seconds. Conditions that vary from the exemplary conditions include, for example, Primer concentrations from about 20 mM to 100 mM; Enzyme from about 1 unit to 4 units; PCR Cycling conditions, annealing temperatures from about 49 C -59 C, and denaturing, annealing, and elongation time from about 30 seconds-2 minutes. Of course, the skilled artisan recognizes that the conditions will depend upon a number of factors including, for example, the number of oligonucleotides and primers used, their length and the extent of complementarity. Those skilled in the art can determine appropriate conditions in view of the extensive knowledge in the art regarding the factors that affect PCR (see, e.g., Molecular Cloning: A Laboratory Manual 3^{rd} ed., Joseph Sambrook, et al., Cold Spring Harbor Laboratory Press; (2001); Short Protocols in Molecular Biology 4^{th} ed., Frederick M. Ausubel (ed.), et al., John Wiley & Sons; (1999); and Pcr (Basics: From Background to Bench) 1^{st} ed., M. J. McPherson, et al., Springer Verlag (2000)).

As used herein, the term “incapable of specifically hybridizing to a sample” and grammatical variants thereof, when used in reference to an oligonucleotide or a primer, means that the oligonucleotide or primer does not specifically hybridize to the sample (e.g., a nucleic acid sample) to the extent that any non-specific hybridization occurring between one or more oligonucleotides or primers and the nucleic acid sample does not interfere with developing the code. Thus, for example where a sample is human nucleic acid, typically all or a part of the oligonucleotide sequence will be non-human (e.g., bacterial, viral, yeast, etc.) such that any non-specific hybridization occurring between one or more oligonucleotides or primers and the human nucleic acid does not interfere with oligonucleotide detection/identification, i.e., identifying the code.

There may be situations where an oligonucleotide or a primer specifically hybridizes to a sample and some amplification of the sample may occur thereby producing a false positive. However, rarely if ever will the size of the false product be the expected size of an oligonucleotide that is a part of the code. Furthermore, a threshold level can be set such that the amount of an oligonucleotide must be greater than a certain threshold in order for the oligonucleotide to be considered “present” or “positive.” If the amount of the oligonucleotide or amplified product produced is greater than the threshold level then the product is considered present. In contrast, if the amount is less than the threshold, then the oligonucleotide or amplified product is considered a false positive. Visual inspection of relative amounts or other quantification means using densitometers or gel scanners can be used to determine whether or not a given product is above or below a certain threshold.

Accordingly, oligonucleotide(s) and primer(s) that specifically hybridize to each other can be entirely non-complementary to a sample that is nucleic acid, or have some or 100% complementarity, provided that any hybridization occurring between the oligonucleotide(s) or primer(s) and the nucleic acid sample does not interfere with developing the code. It is therefore intended that the meaning of “incapable of specifically hybridizing to a sample” used herein includes situations where an oligonucleotide or a primer specifically hybridizes to a sample and amplification of the sample may occur, but the amplification does not interfere with developing the code.

In addition, when there is nucleic acid present in the sample that is ancillary to the sample, that is, for a protein sample or any other non-nucleic acid sample in which nucleic acid happens to be present but is not the sample that is coded, an oligonucleotide or primer may also specifically hybridize to the nucleic acid provided that the hybridization with the nucleic acid sample does not interfere with developing the code. Because the size of any amplified product produced will not have the expected size of the oligonucleotide, such hybridization will rarely if ever interfere with developing the code. Furthermore, in a situation where there is nucleic acid ancillary to the sample, typically the amount of primer(s) is in excess of the nucleic acid such that no interference with developing the code occurs.

Thus, in particular embodiments of the invention, the oligonucleotide(s) or primer(s) will have less than about 40-50% homology with a sample that is nucleic acid. In additional specific embodiments, the oligonucleotide(s) will have less that about 0.5-50% homology, e.g., 45%, 40%, 35%, 30%, 25%, 20%, 15%, 10%, 5%, 3%, or less homology with a sample that is nucleic acid.

The oligonucleotides used for coding the sample may be of any length. For example, oligonucleotides can range in length from 8-10 nucleotides to about 100 Kb in length. In specific embodiments, the oligonucleotides have a length from about 10 nucleotides to about 50 Kb, from about 10 nucleotides to about 25 Kb, from about 10 nucleotides to about 10 Kb, from about 10 nucleotides to about 5 Kb; from about 12 nucleotides to about 1000 nucleotides, from about 15 nucleotides to about 500 nucleotides, from about 20 nucleotides to 250 nucleotides, or from about 25 to 250 nucleotides, 30 to 250 nucleotides, 35 to 200 nucleotides, 40 to 150 nucleotides, 40 to 100 nucleotides, or 50 to 90 nucleotides.

Where the physical difference used for oligonucleotide identification is length, the length differs by at least one nucleotide. Typically, oligonucleotides will differ in sequence length from each other, for example, by 1 to 500, 1 to 300, 1 to 200, 3 to 200, 5 to 150, 5 to 120, 5 to 100, 5 to 75, or 5 to 50 nucleotides; or 2-5, 5-10, 10-20, 20-30, 30-50, 50-100, 100-250, 250-500 or more nucleotides. More typically, the length difference can be in a range convenient for size-fractionation via gel-electrophoresis, for example, 5, 10, 15, 20, 25, 30, 35, 40, 45, 50 nucleotide lengths are convenient to detect differences in the size of oligonucleotides having a length a range from about 20 to 5000 nucleotides.

In the exemplary illustration, the oligonucleotides are amplified and subsequently fractionated via gel electrophoresis. The code however may be developed by any other means capable of differentiating between the oligonucleotides comprising the code. For example, the oligonucleotides whether amplified or not may be fractionated by size-exclusion, paper or ion-exchange chromatography, or be separated on the basis of charge, solubility, diffusion or adsorption. Thus, the means of identifying the oligonucleotides of the code include any method which differentiates between oligonucleotides that may be present in the code.

For example, oligonucleotides having a chemical or physical difference that cannot be differentiated by size-fractionation or differential primer hybridization may be differentiated by other means including modifying the oligonucleotides. As set forth in detail below, oligonucleotides may be labeled using any of a variety of detectable moieties in order to differentiate them from each other. As such, a code may include one or more oligonucleotides that have an identical nucleotide sequence or length but that have some other chemical or physical difference between them that allows them to be distinguished from each other. Accordingly, such oligonucleotides, which may be included in a code as set forth herein, need not be subject to hybridization or subsequent amplification in order to determine identity.

As used herein, the term “different sequence,” when used in reference to oligonucleotides, means that the nucleotide sequences of the oligonucleotides are different from each other to the extent that the oligonucleotides can be differentiated from each other. The different sequence of an oligonucleotide “capable of specifically hybridizing to a unique primer pair” therefore includes any contiguous sequence that is suitable for primer hybridization such that the oligonucleotide can be differentiated on the basis of differential primer hybridization from other oligonucleotides potentially present. The oligonucleotides will differ in sequence from each other by at least one nucleotide, but typically will exhibit greater differences to minimize non-specific hybridization, e.g., 2-5, 5-10, 10-20, 20-30, 30-50, 50-100, 100-250, 250-500 or more nucleotides in the oligonucleotides will differ from the other oligonucleotides. The number of nucleotide differences to achieve differential primer hybridization and, therefore, oligonucleotide differentiation will be influenced by the size of the oligonucleotide, the sequence of the oligonucleotide, the assay conditions (e.g., hybridization conditions such as temperature and the buffer composition), etc. Oligonucleotide sequence differences may also be expressed as a percentage of the total length of the oligonucleotide sequence, e.g., when comparing the two oligonucleotides, the percentage of the nucleotides that are either identical or different from each other. Thus, for example, for a 30 bp oligonucleotide (OL1) as little as 20-25% of the sequence need be different from another oligonucleotide sequence (OL2) in order to differentiate between OL1 and OL2, provided that the sequences of OL1 and OL2 that are 75-80% identical do not interfere with developing the code.

The term “different sequence,” when used in reference to oligonucleotides, refers to oligonucleotides in which differential primer hybridization is used to differentiate among the oligonucleotides comprising the code. This does not preclude the presence of other oligonucleotides in the code where differential primer hybridization is not used to identify them. For example, two or more oligonucleotides of the code can have an identical nucleotide sequence where a primer pair hybridizes. Thus, such oligonucleotides are not distinguished from each other on the basis of length or differential primer hybridization. However, oligonucleotides having the same primer hybridization sequence can have different sequence length, or some other physical or chemical difference such as charge, solubility, diffusion adsorption or a label, such that they can be differentiated from each other on the basis of size. Accordingly, oligonucleotides of the code can have the same nucleotide sequence where a primer pair hybridizes and as such, a primer pair can specifically hybridize to two or more oligonucleotides of the code.

The oligonucleotide sequence determines the sequence of the primer pairs used to detect the oligonucleotides. As disclosed herein, using unique primer pairs that specifically hybridize to each of the oligonucleotides potentially present in a query sample facilitates detection of all oligonucleotides. Typically, the corresponding primer pairs hybridize to a portion of the oligonucleotide sequence. Thus, the sequence region to which the primers hybridize is the only nucleotide sequence that need be known in order to detect the oligonucleotide. In other words, in order to detect or identify any oligonucleotide of the code, only the nucleotide sequence that participates in primer hybridization needs to be known. Accordingly, nucleotide sequences of an oligonucleotide that do not participate in specific hybridization with a primer pair can be any sequence or unknown.

For example, where the primer pairs hybridize at the 5′ or 3′ end of an oligonucleotide, the intervening sequence between the hybridization sites can be any sequence or can be unknown. Likewise, for primer pairs that hybridize near the 5′ or 3′ end of an oligonucleotide, the intervening sequence between the primer hybridization sites or the sequences that flank the primer hybridization sites can be any sequence or can be unknown. In either case, nucleotides located between or that flank primer hybridization sites can be any sequence or unknown, provided that the intervening or flanking sequences do not hybridize to different oligonucleotides, non-target primers or to a sample that is nucleic acid to such an extent that it interferes with developing the code.

Since the nucleotide sequence of the oligonucleotides to which the primers hybridize confer hybridization specificity which in turn indicates the identity of the oligonucleotide (e.g., OL1), nucleotides that do not participate in primer hybridization may be identical to nucleotides in different oligonucleotides (e.g., OL2) that do not participate in primer hybridization. For example, if a particular oligonucleotide is 30 nucleotides in length (OL1), a primer could be as few as 8 nucleotides meaning that 14 nucleotides in the oligonucleotide are not participating in primer hybridization. Thus, all or a part of these 14 contiguous nucleotides in OL1 can be identical to one or more of the other oligonucleotides in the same set or in a different set (e.g., OL2, OL3, OL4, OL5, OL6, etc.), provided that the primer pairs that specifically hybridize to OL2, OL3, OL4, OL5, OL6, etc., do not also hybridize to this 14 nucleotide sequence to the extent that this interferes with developing the code. Accordingly, nucleotide sequences regions within oligonucleotide that do not participate in primer hybridization may be identical to each other in part or entirely.

The location of the different sequence capable of specifically hybridizing to a unique primer pair in an oligonucleotide will typically be at or near the 5′ and 3′ termini of the oligonucleotide. The location of the different sequence capable of specifically hybridizing to a unique primer pair in the oligonucleotide is influenced by oligonucleotide length. For example, for shorter oligonucleotides the location of the different sequence capable of specifically hybridizing to a unique primer pair is typically at or near the 5′ and 3′ termini. In contrast, with longer oligonucleotides the location of the different sequence capable of specifically hybridizing to a unique primer pair can be further away from the 5′ and 3′ termini. Where oligonucleotide size differences are used for identification, there need only be size differences between the oligonucleotides in the code or in the amplified oligonucleotide products. Thus, if the oligonucleotides are detected in the absence of amplification, the sizes of the oligonucleotides will be different from each other. In contrast, if amplification is used to develop the code as in the exemplary illustration, the primers in a given set need only specifically hybridize to the oligonucleotides in the set (i.e., not at the 5′ and 3′ termini) to produce amplified products having different sizes from each other. In other words, oligonucleotides within a given set can have an identical length provided that the primers specifically hybridize with the oligonucleotide at locations that produce amplified products having a different size. As an example, two oligonucleotides, OL1 and OL2, within a given set each have a length of 50 nucleotides. When developing the code primer pairs that specifically hybridize at the 5′ and 3′ termini of OL1 produce an amplified product of 50 nucleotides, whereas primer pairs that specifically hybridize 5 nucleotides within the 5′ and 3′ termini of OL2 produce an amplified product of 40 nucleotides.

Thus, the location of the different sequence capable of specifically hybridizing to a unique primer pair in an oligonucleotide can, but need not be, at the 5′ and 3′ termini of the oligonucleotide. In one embodiment, the different sequence is located within about 0 to 5, 5 to 10, 10 to 25 nucleotides of the 3′ or 5′ terminus of the oligonucleotide. In another embodiment, the different sequence is located within about 25 to 50 or 50 to 100 nucleotides of the 3′ or 5′ terminus of the oligonucleotide. In additional embodiments, the different sequence is located within about 100 to 250, 250 to 500, 500 to 1000, or 1000 to 5000 nucleotides of the 3′ or 5′ terminus of the oligonucleotide.

As used herein, the terms “oligonucleotide,” “nucleic acid,” “polynucleotide,” “primer,” and “gene” include linear oligomers of natural or modified monomers or linkages, including deoxyribonucleotides, ribonucleotides, and α-anomeric forms thereof capable of specifically hybridizing to a target sequence by way of a regular pattern of monomer-to-monomer interactions, such as Watson-Crick type of base pairing, base stacking, Hoogsteen or reverse Hoogsteen types of base pairing. Monomers are typically linked by phosphodiester bonds or analogs thereof to form the polynucleotides. Oligonucleotides can be a synthetic oligomer, a sense or antisense, circular or linear, single, double or triple strand DNA or RNA. Whenever an oligonucleotide is represented by a sequence of letters, such as “ATGCCTG,” the nucleotides are in a 5′ to 3′ orientation from left to right.

Essentially any polymer that has a unique sequence can be used for the code, provided the polymer is detectable and can be distinguished from other polymers present in the code. Polymers include organic polymers or alkyl chains identified by spectroscopy, e.g., NMR and FT-IR. Polymers include one or more amino acids attached thereto, for example, peptides derivatized with ninhydrin or opthaldehyde, which can be detected with a fluorometer. Polymers further include peptide nucleic acid (PNA), which refers to a nucleic acid mimic, e.g., DNA mimic, in which the deoxyribose phosphate backbone is replaced by a pseudopeptide backbone while retaining the natural nucleotides.

Oligonucleotides therefore include moieties which have all or a portion similar to naturally occurring oligonucleotides but which are non-naturally occurring. Thus, oligonucleotides may have one or more altered sugar moieties or inter-sugar linkages. Particular examples include phosphorothioate and other sulfur-containing species known in the art. One or more phosphodiester bonds of the oligonucleotide can be substituted with a structure that enhances stability of the oligonucleotide. Particular non-limiting examples of such substitutions include phosphorothioate bonds, phosphotriesters, methyl phosphonate bonds, short chain alkyl or cycloalkyl structures, short chain heteroatomic or heterocyclic structures and morpholino structures (U.S. Pat. No.5,034,506). Additional linkages include are disclosed in U.S. Pat. Nos. 5,223,618 and 5,378,825.

Oligonucleotides therefore further include nucleotides that are naturally occurring, synthetic, and combinations thereof. Naturally occurring bases include adenine, guanine, cytosine, thymine, uracil and inosine. Particular non-limiting examples of synthetic bases include xanthine, hypoxanthine, 2-aminoadenine, 6-methyl, 2-propyl and other alkyl adenines, 5-halo uracil, 5-halo cytosine, 6-aza cytosine and 6-aza thymine, psuedo uracil, 4-thiuracil, 8-halo adenine, 8-aminoadenine, 8-thiol adenine, 8-thioalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil, 5-trifluoro cytosine and tritylated bases.

Oligonucleotides can be made nuclease resistant during or following synthesis in order to preserve the code. Oligonucleotides can be modified at the base moiety, sugar moiety or phosphate backbone to improve stability, hybridization, or solubility of the molecule. For example, the 5′ end of the oligonucleotide may be rendered nuclease resistant by including one or more modified intenucleotide linkages (see, e.g., U.S. Pat. No. 5,691,146).

The deoxyribose phosphate backbone of oligonucleotide(s) can be modified to generate Peptide nucleic acids (Hyrup et al., Bioorg. Med. Chem. 4:5 (1996)). The neutral backbone of PNAs allows specific hybridization to DNA and RNA under conditions of low ionic strength. The synthesis of PNA oligomers can be performed using standard solid phase peptide synthesis protocols (see, e.g., Perry-O'Keefe et al., Proc. Natl. Acad. Sci. USA 93:14670 (1996)). PNAs hybridize to complementary DNA and RNA sequences in a sequence-dependent manner, following Watson-Crick hydrogen bonding. PNA-DNA hybridization is more sensitive to base mismatches; PNA can maintain sequence discrimination up to the level of a single mismatch (Ray and Bengt, FASEB J. 14:1041 (2000)). Due to the higher sequence specificity of PNA hybridization, incorporation of a mismatch in the duplex considerably affects the thermal melting temperature. PNA also be modified to include a label, and the labeled PNA included in the code or used as a primer or probe to detect the labeled PNA in the code. For example, a PNA light-up probe in which the asymmetric cyanine dye thiazole orange (TO) has been tethered. When the light-up PNA hybridizes to a target, the dye binds and becomes fluorescent (Svavnik et al., Analytical Biochem. 281:26 (2000)).

Compositions of the invention including oligonucleotides can include additional components or agents that increase stability or inhibit degradation of the oligonucleotides, i.e., a preservative. Particular non-limiting examples of preservatives include, for example, EDTA, EGTA, guanidine thiocyanate and uric acid.

As used herein, the term “unique primer pair” means a primer pair that specifically hybridizes to an oligonucleotide target under the conditions of the assay. As disclosed herein, a primer pair may hybridize to two or more oligonucleotides that are potentially present in the code. A unique primer pair need only be complementary to at least a portion of the target oligonucleotide such that the primers specifically hybridize and the code is developed. For example, oligonucleotide sequences from about 8 to 15 nucleotides are able to tolerate mismatches; the longer the sequence, the greater the number of mismatches that may be tolerated without affecting specific hybridization. Thus, an 8 to 15 base sequence can tolerate 1-3 mismatches; a 15 to 20 base sequence can tolerate 1-4 mismatches; a 20 to 25 base sequence can tolerate 1-5 mismatches; a 25 to 30 base sequence can tolerate 1-6 mismatches, and so forth.

The hybridization is specific in that the primer pair does not significantly hybridize to non-target oligonucleotides, other primers or a sample that is nucleic acid to an extent that interferes with developing the code. Thus, primer pairs can share partial complementary with non-target oligonucleotides because stringency of the hybridization or amplification conditions can be such that the primer pairs preferentially hybridize to a target oligonucleotide(s). For example, in the case of a 30 base oligonucleotide, OL1, with 10 base primer pairs (Primers#1 and #2), and a 40 base oligonucleotide, OL2, with 10 base primer pairs (Primers#3 and #4), Primers #1 and #3 and/or Primers #2 and #4 can share sequence identity, for example, from 1 to about 5 contiguous nucleotides may be identical between Primers #1 and #3 and/or Primers #2 and #4 without interfering with developing the code. As primer length increases the number of contiguous nucleotides that may be non-complementary with a target oligonucleotide increases. As primer length increases the number of contiguous nucleotides that may be complementary with a non-target oligonucleotide or another primer likewise increases. Generally, the maximum number of contiguous nucleotides that may be identical between primers targeted to different oligonucleotides without interfering with developing the code will be about 40-60%. In any event, the primers need not be 100% homologous to or have 100% complementary with the target oligonucleotides.

Primer pairs can be any length provided that they are capable of hybridizing to the target oligonucleotide and, where amplification is used to develop the code, capable of functioning as a primer for oligonucleotide amplification. In particular embodiments of the invention, one or more of the primers of the unique primer pairs has a length from about 8 to 250 nucleotides, e.g., a length from about 10 to 200, 10 to 150, 10 to 125, 12 to 100, 12 to 75, 15 to 60, 15 to 50, 18 to 50, 20 to 40, 25 to 40 or 25 to 35 nucleotides. In additional embodiments of the invention, one or more of the primers of the unique primer pairs has a length of about 9/10, 4/5, 3/4, 7/10, 3/5, 1/2, 2/5, 1/3, 3/10, 1/4, 1/5, 1/6, 1/7, 1/8, 1/10 of the length of the oligonucleotide to which the primer binds.

Individual primers in a primer pair, primer pairs in a primer set and primers of different sets can have the same or different lengths. In particular embodiments of the invention, each primer of a given unique primer pair, each primer pair in a primer set and primers in different primer sets have the same length or differ in length from about 1 to 500, 1 to 250, 1 to 100, 1 to 50, 1 to 25, 1 to 10, or 1 to 5 nucleotides.

In the exemplary illustration, the code is developed by specific hybridization to primers and subsequent amplification and size-fractionation of the oligonucleotides that hybridize to the primers via electrophoresis. In addition to alternative ways of size-fractionation of the oligonucleotides, which include, size-exclusion, ion-exchange, paper and affinity chromatography, diffusion, solubility, adsorption, there are alternative methods of code development. For example, oligonucleotides could be amplified, then subsequently cleaved with an enzyme to produce known fragments with known lengths that could be the basis for a code. Alternatively, if a sufficient amount of oligonucleotide is present, the oligonucleotides may be size-fractionated without hybridization and subsequent amplification and directly visualized (e.g., electrophoretic size fractionation followed by UV fluorescence). Thus, the oligonucleotide(s) can be detected and, therefore, the code developed without hybridization or amplification.

Another way of detecting the oligonucleotides of the code without hybridization or amplification and, furthermore, without the oligonucleotides having a different length or primer hybridization sequence, is to physically or chemically modify one or more of the oligonucleotides. For example, oligonucleotides can be modified to include a molecular beacon. One specific example is the stem-loop beacon where in the absence of hybridization, the oligonucleotide forms a stem-loop structure where the 5′ and 3′ termini comprise the stem, and the beacon (fluorophore, e.g., TMR) located at one termini of the stem is close to the quencher (e.g., DABCYL-CPG) located at the other termini of the stem. In this stem-loop configuration the beacon is quenched and, therefore, there is no emission by the oligonucleotide. When the oligonucleotide hybridizes to a complementary nucleic acid the stem structure is disrupted, the fluorophore is no longer quenched and the oligonucleotide then emits a fluorescent signal (see, e.g., Tan et al., Chem. Eur. J. 6:1107 (2000)). Thus, by including different beacons in oligonucleotides having different emission spectrums, each oligonucleotide containing a unique beacon can be identified by merely detecting the emission spectrum, without amplification or size-fractionation. Another specific example is the scorpion-probe approach, in which the stem-loop structure with the beacon and quencher is incorporated into a primer. When the primer hybridizes to the target oligonucleotide and the target is amplified, the primer is extended unfolding the stem-loop and the loop hybridizes intramolecularly with its target sequence, and the beacon emits a signal (see, e.g., Broude, N. E. Trends Biotechnol. 20:249 (2002)). As the number of beacons expands, the number of unique codes available expands. Thus, beacons in oligonucleotides can be used in combination with other oligonucleotides having a physical or chemical difference of the code, such as a different length.

Additional physical or chemical modifications that facilitate developing the code without amplification or fractionation include radioisotope-labeled nucleotides (e.g., dCTP) and fluorescein-labeled nucleotides (UTP or CTP). Detecting the labels indicates the presence of the oligonucleotide so labeled. The labels may be incorporated by any of a number of means well known to those skilled in the art. For example, the oligonucleotides can be directly labeled without hybridization or amplification or during oligonucleotide amplification, in which case the oligonucleotide(s) primer pairs can be labeled before, during, or following hybridization and subsequent amplification. Typically labeling occurs before hybridization. In a particular example, PCR with labeled primers or labeled nucleotides will produce a labeled amplification product.

“Direct labels” are directly attached to or incorporated into the oligonucleotides prior to hybridization. Alternatively, a label may be attached directly to the primer or to the amplification product after the amplification is completed using methods well known to those of skill in the art including, for example nick translation or end-labeling. Indirect labels are attached to the hybrid duplex after hybridization. For example, an indirect label such as biotin can be attached to the oligonucleotides prior to hybridization. Following hybridization, an avidin-conjugated fluorophore will bind the biotin bearing hybrid duplexes to facilitate detection of the oligonucleotide.

Labels therefore include any composition that can be attached to or incorporated into nucleic acid that is detectable by spectroscopic, photochemical, biochemical, immunochemical, electrical, optical or chemical means such that it provides a means with which to identify the oligonucleotide. Useful labels include biotin for staining with labeled streptavidin conjugate, magnetic beads (e.g., Dynabeads™), fluorescent dyes (e.g., 6-FAM, HEX, TET, TAMRA, ROX, JOE, 5-FAM, R110, fluorescein, texas red, rhodamine, lissamine, phycoerythrin (Perkin Elmer Cetus), Cy2, Cy3, Cy3.5, Cy5, Cy5.5, Cy7, FluorX (Amersham Biosciences; Genisphere, Hatfield, Pa.), radiolabels, enzymes (e.g., horse radish peroxidase, alkaline phosphatase and others used in ELISA), Alexa dyes (Molecular Probes), Q-dots and colorimetric labels, such as colloidal gold or colored glass or plastic beads (e.g., polystyrene, polypropylene, latex, etc.).

When the code is developed in the exemplary illustration, the oligonucleotides are mixed with primer sets. Thus, the invention further provides compositions including a plurality of unique primer pairs (e.g., two or more) and a plurality of oligonucleotides (e.g., two or more) with or without a sample.

The unique primer pairs are within a given primer set. That is, whether or not one or more of the individual oligonucleotides of a code are present, the primer pairs are capable of specifically hybridizing to and amplifying one or more oligonucleotides of the code. If present, oligonucleotides differentiated by size will be amplified and the amplified products will have different lengths. In various embodiments, a composition includes three or more unique primer pairs and two or more oligonucleotides, wherein the unique primer pairs are denoted a first, second, third, fourth, fifth, sixth, etc., primer set, one or more of the unique primer pairs having a different sequence, at least two of the unique primer pairs capable of specifically hybridizing to the two oligonucleotides. The corresponding oligonucleotides to which the primers hybridize are denoted a first, second, third, fourth, fifth, sixth, etc. oligonucleotide set, the oligonucleotides having a length from about 8 nucleotides to 50 Kb, the oligonucleotides in each set having a physical or chemical difference (e.g., a different length) from the other oligonucleotides comprising the same oligonucleotide set. In various aspects, the number of primer pairs in a set is four or more, five or more, six or more unique primer pairs (e.g., seven, eight, nine, ten, 11, 12, 13, 14, 15, 15-20, 20-25, and so on and so forth). In various additional aspects, the number of oligonucleotides is three, four, five, six or more (e.g., seven, eight, nine, ten, 11, 12, 13, 14, 15, 15-20, 20-25, and so on and so forth).

In additional embodiments, compositions include one or more oligonucleotides denoted a second oligonucleotide set, each of the oligonucleotides having a different sequence therein capable of specifically hybridizing to a unique primer pair, the unique primer pair from a second primer set. The second oligonucleotide set includes oligonucleotides incapable of specifically hybridizing to a sample, a length from about 8 nucleotides to 50 Kb, and a physical or chemical difference (e.g., a different length) from the other oligonucleotides within the second oligonucleotide set. In one aspect, one or more oligonucleotides of the second oligonucleotide set have the same length as an oligonucleotide of the first oligonucleotide set. In farther embodiments, compositions include one or more oligonucleotides denoted a third oligonucleotide set, each of the oligonucleotides having a different sequence therein capable of specifically hybridizing to a unique primer pair, the unique primer pair from a third primer set. The third oligonucleotide set includes oligonucleotides incapable of specifically hybridizing to a sample, a length from about 8 nucleotides to 50 Kb, and a physical or chemical difference (e.g., a different length) from the oligonucleotides within the third oligonucleotide set. In farther aspects, one or more oligonucleotides of the third oligonucleotide set has the same length as an oligonucleotide of the first or second oligonucleotide set.

Invention compositions can include one or more additional oligonucleotide sets (e.g., fourth, fifth, sixth, seventh, eighth, ninth, tenth, etc. sets), the additional oligonucleotide sets each including oligonucleotides within that set having a different sequence therein capable of specifically hybridizing to a unique primer pair from a corresponding primer set (e.g., fourth, fifth, sixth, seventh, eighth, ninth, tenth, etc. sets). Each oligonucleotide within each of the additional oligonucleotide sets is incapable of specifically hybridizing to a sample, has a length from about 8 nucleotides to 50 Kb, and has a physical or chemical difference (e.g., a different length) from the other oligonucleotides within that oligonucleotide set.

As used herein, the term “sample” means any physical entity, which is capable of being coded in accordance with the invention. Samples therefore include any material which is capable of having a code associated with the sample. A sample therefore may include non-biological and biological samples as well as samples suitable for introduction into a biological system, e.g., prescription or over-the-counter medicines (e.g., pharmaceuticals), cosmetics, perfume, foods or beverages.

Specific non-limiting examples of non-biological samples include documents, such as letters, commercial paper, bonds, stock certificates, contracts, evidentiary documents, testamentary devices (e.g., wills, codicils, trusts); identification or certification means, such as birth certificates, licensing certificates, signature cards, driver's licenses, identification cards, social security cards, immigration status cards, passports, fingerprints; negotiable instruments, such as currency, credit cards, or debit cards. Additional non-limiting examples of non-biological samples include wearable garments such as clothing and shoes; containers, such as bottles (plastic or glass), boxes, crates, capsules, ampoules; labels, such as authenticity labels or trademarks; artwork such as paintings, sculpture, rugs and tapestries, photographs, books; collectables or historical or cultural artifacts; recording medium such as analog or digital storage medium or devices (e.g., videocassette, CD, DVD, DV, MP3, cell phones); electronic devices such as, instruments; jewelry such as rings, watches, bracelets, earrings and necklaces; precious stones or metals such as diamonds, gold, platinum; and dangerous devices, such as firearms, ammunition, explosives or any composition suitable for preparing explosives or an explosive device.

Specific non-limiting examples of biological samples include foods, such as meat (e.g., beef, pork, lamb, fowl or fish), grains and vegetables; and alcohol or non-alcoholic beverages, such as wine. Non-limiting examples of biological samples also include tissues and whole organs or samples thereof, forensic samples and biological fluids such as blood (blood banks), plasma, serum, sputum, semen, urine, mucus, stool and cerebrospinal fluid. Additional non-limiting examples of biological samples include living and non-living cells, eggs (fertilized or unfertilized) and sperm (e.g., animal husbandry or breeding samples). Further non-limiting examples of biological samples include bacteria, virus, yeast, or mycoplasma, such as a pathogen (e.g., smallpox, anthrax).

Samples that are nucleic acid include mammalian (e.g., human), bacterial, viral, archaea and fungi (e.g., yeast) nucleic acid. As discussed, oligonucleotides used to code such nucleic acid samples do not specifically hybridize to the nucleic acid sample to the extent that the hybridization interferes with developing the code. Thus, for example, where the sample is human nucleic acid, the oligonucleotides typically do not specifically hybridize to the human nucleic acid; where the sample is bacterial nucleic acid, the oligonucleotides typically do not specifically hybridize to the bacterial nucleic acid; where the sample is viral nucleic acid, the oligonucleotides typically do not specifically hybridize to the viral nucleic acid, etc.

The association between the code and the sample is any physical relationship in which the code is able to uniquely identify the sample. The code may therefore be attached to, integrated within, impregnated with, mixed with, or in any other way associated with the sample. The association does not require physical contact between the code and the sample. Rather, the association is such that that the sample is identified by the code, whether the sample and code physically contact each other or not. For example, a code may be attached to a container (e.g., a label on the outside surface of a vial) which contains the sample within. A code can be associated with product packaging within which is the actual sample. A code can be attached to a housing or other structure that contains or otherwise has some association with the sample such that the code is capable of uniquely identifying the sample, without the code actually physically contacting the sample. The code and sample therefore do not need to physically contact each other, but need only have a relationship where the code is capable of identifying the sample.

Oligonucleotides can be added to or mixed with the sample and the mixture can be a solid, semi-solid, liquid, slurry, dried or desiccated, e.g., freeze-dried. Oligonucleotides can be relatively inseparable from the sample. For example, where the oligonucleotides are mixed with a sample that is a biological sample such as nucleic acid, the oligonucleotides are separable from the sample using a molecular biological or, biochemical or biophysical technique, such as size- or affinity based electrophoresis, column chromatography, hybridization, differential elution, etc. As set forth herein, oligonucleotides can be in a relationship with the sample such that they are easily physically separable from the sample. In the example of a substrate, one or more of the oligonucleotides can be easily physically separable from the sample, under conditions where the sample remains substantially attached to the substrate. For example, when the oligonucleotides are affixed to a dry solid medium (e.g., Guthrie card) and the sample is likewise affixed to the same dry solid medium, the two may be affixed at different positions on the medium. By knowing the position of the oligonucleotides or sample, they can be easily physically separated by removing a section of the substrate to which the oligonucleotides or sample are attached (e.g., a punch). In another example, the oligonucleotides may be dispensed in a well of a multi-well plate (e.g., 96 well plate), with other wells of the plate containing sample(s). The oligonucleotides are physically separated from the sample by retrieving them from the well (e.g., with a pipette) into which they were dispensed.

In either case, whether oligonucleotides of the code physically contact the sample, or the oligonucleotides of the code are associated with but do not physically contact the sample, the oligonucleotides can be identified in order to develop the code. Thus, the invention is not limited with respect to the nature of the association between the oligonucleotides of the code and the sample that is coded.

Substrates to which the oligonucleotides and samples can be affixed, attached or stored within or upon include essentially any physical entity such as two dimensional surface that is permeable, semi-permeable or impermeable, either rigid or pliable and capable of either storing, binding to or having attached thereto or impregnated with oligonucleotides. Substrates include dry solid medium (e.g., cellulose, polyester, nylon, or mixtures thereof etc.). Specific commercially available dry solid medium includes, for example, Guthrie cards, IsoCode (Schleicher and Schuell), and FTA (Whatman). A medium having a mixture of cellulose and polyester is useful in that low molecular weight nucleic acid (e.g., the oligonucleotides comprising the code) preferentially binds to the cellulose component and high molecular weight nucleic acid (e.g., genomic DNA) preferentially binds to the polyester component. A specific example of a cellulose/polyester blend is LyPore SC (Lydall), which contains about 10% cellulose fiber and 90% polyester. Washing the dry solid medium with an appropriate liquid or removing a section (e.g., a punch) retrieves the oligonucleotides or sample from the medium, which can subsequently be analyzed to develop the code or to analyze the sample.

Substrates include foam, such as an absorbent foam. In the particular example of a sponge-like absorbent foam having oligonucleotides or sample, the foam can be wet or wetted with an appropriate liquid, and squeezed or centrifuged to release liquid containing the oligonucleotides or sample. Substrates include structures having sections, compartments, wells, containers, vessels or tubes, separated from each other to prevent mixing of samples with each other or with the oligonucleotides. Multi-well plates, which typically contain 6 to 1000 wells, are one particular non-limiting example of such a structure.

Substrates also include supports used for two- or three-dimensional arrays of nucleic acid or protein sequences. The nucleic acid or protein sequences (e.g., sample(s)) are typically attached to the surface of the substrate (e.g., via a covalent bond) at defined positions (addresses). Substrates can include a number of nucleic acid or protein sequences greater than about 25, 50, 100, 1000, 10,000, 100,000, 1,000,000, or more. Such substrates, also referred to as “gene chips” or “arrays,” can have any nucleic acid or protein density; the greater the density the greater the number of sequences that can be screened on a given chip. Substrates that include a two- or three-dimensional array of nucleic acid or protein sequences, and individual nucleic acid or protein sequences therein, may be coded in accordance with the invention.

For example, the substrate itself can be the sample, in which case a substrate containing a plurality of nucleic acid or protein sequences will have a unique code. Alternatively, one or more of each individual nucleic acid or protein sequence on the substrate can have an individual code. For example, a unique oligonucleotide code can be added to one or more samples on the substrate in order to uniquely identify the coded samples.

The invention provides kits including compositions as set forth herein. In one embodiment, a kit includes two or more oligonucleotides in one or more oligonucleotide sets, packaged into suitable packaging material. Kits can contain oligonucleotide(s) of one or more sets, primer pair(s) of one or more sets, optionally alone or in combination with each other. A kit typically includes a label or packaging insert including a description of the components or instructions for use (e.g., coding a sample). A kit can contain additional components, for example, primer pairs that specifically hybridize to the oligonucleotides.

The term “packaging material” refers to a physical structure housing the components of the kit. The packaging material can maintain the components sterilely, and can be made of material commonly used for such purposes (e.g., paper, corrugated fiber, glass, plastic, foil, ampoules, etc.). The label or packaging insert can include appropriate written instructions, for example, practicing a method of the invention. Kits of the invention therefore can additionally include labels or instructions for using the kit components in a method of the invention. Instructions can include instructions for practicing any of the methods of the invention described herein. The instructions may be on “printed matter,” e.g., on paper of cardboard within the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, DV, MP3, magnetic tape, electrical storage media such as RAM and ROM and hybrids of these such as magnetic/optical storage media.

Invention kits can include each component (e.g., the oligonucleotides) of the kit enclosed within an individual container and all of the various containers can be within a single package. Invention kits can be designed for long-term, e.g., cold storage.

The invention provides methods of producing samples that are coded (i.e., “bio-tagged”) in order to identify the sample. In one embodiment, a method includes: selecting a combination of two or more oligonucleotides to add to the sample which are incapable of specifically hybridizing to the sample, each having a length from about 8 to 50 Kb nucleotides and a physical or chemical difference (e.g., a different length), and one or more having a different sequence therein capable of specifically hybridizing to a unique primer pair; and adding the combination of two or more oligonucleotides to the sample. The combination of oligonucleotides identifies the sample and, therefore, the method produces a bio-tagged sample. In additional embodiments, a method of the invention employs one or more oligonucleotides from multiple (e.g., two, three, four, five, six, seven, eight, nine, ten, etc., or more) oligonucleotide sets in which one or more oligonucleotides from the additional oligonucleotide sets is added to the sample. In one particular embodiment, one or more oligonucleotides from a second set is added, one or more of the oligonucleotide(s) of the second set having a different sequence therein capable of specifically hybridizing to a unique primer pair of a second primer set, incapable of specifically hybridizing to the sample, a physical or chemical difference (e.g., a different length) from the other oligonucleotides of the second set, and a length from about 8 to 50 Kb nucleotides. In another particular embodiment, one or more oligonucleotides from a third oligonucleotide set is added, one or more of the oligonucleotide(s) of the third set having a different sequence therein capable of specifically hybridizing to a unique primer pair of a third primer set, incapable of specifically hybridizing to the sample, a physical or chemical difference (e.g., a different length) from the other oligonucleotides of the third set and a length from about 8 to 50 Kb nucleotides. In one aspect of the methods of producing a coded sample, one or more of the oligonucleotides of the code is physically separated or separable from the sample.

The invention also provides methods of identifying a coded (i.e., “bio-tagged”) sample. In one embodiment, a method includes: detecting in a sample the presence or absence of two or more oligonucleotides, wherein the oligonucleotides are identified based upon a physical or chemical difference (e.g., length), thereby identifying a combination of oligonucleotides in the sample; comparing the combination of oligonucleotides to a database of particular oligonucleotide combinations known to identify particular samples; and identifying the sample based upon which of the particular oligonucleotide combinations in the database is identical to the combination of oligonucleotides in the sample. The oligonucleotide combination can be identified based upon a primer or primer pair(s) that specifically hybridizes to the oligonucleotides, e.g., differential primer hybridization with or without subsequent amplification. Thus, in another embodiment, a method further includes specifically hybridizing one or more unique primer pairs of one or more primer sets to the oligonucleotides that may be present thereby identifying oligonucleotide(s) present. Oligonucleotides are identified based upon primer pair(s) hybridization to the oligonucleotides that are present; the combination of particular oligonucleotides present in the sample is the code of the sample. Methods for identifying/detecting the oligonucleotides include hybridization to two or more unique primer pairs having a different sequence; and hybridization to two or more unique primer pairs having a different sequence and subsequent amplification (e.g., PCR). In further aspects, oligonucleotides that are likely to be present in the sample are selected from two or more oligonucleotide sets (e.g., two, three, four, five, six, seven, eight, nine, etc. sets) and, as such, a method of the invention can additionally include specifically hybridizing one or more unique primer pairs of two or more primer sets to the oligonucleotides that may be present with or without subsequent amplification in order to identify which of the oligonucleotides from the different oligonucleotide sets are present.

The invention further provides archives of coded (i.e., bio-tagged) sample(s). In one embodiment, an archive of bio-tagged samples includes: one or more samples; two or more oligonucleotides incapable of specifically hybridizing to one or more of the samples, the oligonucleotides each having a physical or chemical difference (e.g., a different length), and a length from about 8 to 50 Kb nucleotides, one or more of the oligonucleotides having a different sequence therein capable of specifically hybridizing to a unique primer pair, in a unique combination that identifies the one or more samples; and a storage medium for storing the sample(s). In various aspects, an archive includes 1 to 10, 10 to 50, 50 to 100, 100 to 500, 500 to 1000, 1000 to 5000, 5000 to 10,000, 10,000 to 100,000, or more samples, one or more of which is coded.

The invention further provides methods of producing archives of coded (i.e., bio-tagged) samples. In one embodiment, a method includes: selecting a combination of two or more oligonucleotides that are incapable of specifically hybridizing to the sample, each having a chemical or physical difference (e.g., a different length), and a length from about 8 to 50 Kb nucleotides, and one or more of the oligonucleotides having a different sequence therein capable of specifically hybridizing to a unique primer pair; and adding the combination of two or more oligonucleotides to a sample. The bio-tagged sample produced is then placed in a storage medium. Two or more samples placed in a storage medium comprises an archive.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All publications, patents and other references cited herein are incorporated by reference in their entirety. In case of conflict, the present specification, including definitions, will control.

As used herein, the singular forms “a”, “and,” and “the” include plural referents unless the context clearly indicates otherwise. Thus, for example, reference to “an oligonucleotide or a primer or a sample” includes a plurality of such oligonucleotides, primers and samples, and reference to “an oligonucleotide set” or “a primer set” includes reference to one or more oligonucleotide or primer sets, and so forth.

The invention set forth herein is described with affirmative language. Therefore, even though the invention is generally not expressed herein in terms of what the invention does not include, aspects that are not expressly included in the invention are nevertheless inherently disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLE 1

This example describes an exemplary code using 50, 75 and 100 base oligonucleotides in a single set.

Oligonucleotides comprising the code and corresponding primers were designed by selecting a non-human gene from Genbank, Arabidopsis thaliana lycopene beta cyclase, accession number U50739, using the default settings on the Primer 3 program: http://www-genome.wi.mit.edu/cgi-bin/primer/primer3_www.cgi. In order to multiplex the primers in one reaction, the primer pairs were selected from the output of Primer 3 to have a similar melting temperature. To ensure that the sequences selected do not have a significant match to the reported human genes and EST sequences, a Blast (http://www.ncbi.nlm.nih.gov/BLAST/) comparison was preformed against genbank's non-redundant (nr) database.

50 bp oligonucleotide, PCR primer #1-5′ TCCATCTCCA
TGAAGCTACT 3′
50 bp oligonucleotide, PCR primer #2-5′ ATGAACGAAG
ACCACAAAAC 3′
50 bp oligonucleotide-5′ CCATCTCCATGAAGCTACTGCTTCT
GGGTAAGTTTTGTGGTCTTCGTTCAT 3′
(SEQ ID NOs:1-3, respectively)
75 bp oligonucleotide, PCR primer #1-5′ GTGTCAAGAA
GGATTTGAGC 3′
75 bp oligonucleotide, PCR primer #2-5′ TTTCTGAAGC
ATTTTGGATT 3′
75 bp oligonucleotide -5′ GTGTCAAGAAGGATTTGAGCCGGC
CTTATGGGAGAGTTAACCGGAAACAGCTCAAATCCAAAATGCTTCAGAAA
3′
(SEQ ID NOs:4-6, respectively)
100 bp oligonucleotide, PCR primer #1-5′ TCTGAAGCT
GGACTCTCTGT 3′
100 bp oligonucleotide, PCR primer #2-5′ AATCCATAG
CCTCAAACTCA 3′
100 bp oligonucleotide-5′ TCTGAAGCTGGACTCTCTGTTTG
TTCCATTGATCCTTCTCCTAAGCTCATATGGCCTAACAATTATGGAGTTT
GGGTTGATGAGTTTGAGGCTATGGATT 3′
(SEQ ID NOs:7-10, respectively)

The oligonucleotides were applied to the media in solution. A solution is made up of the desired combination of oligonucleotides at a concentration of 0.1 uM each. Three microliters of the solution is then applied to the media (FTA or Iso-Code) and allowed to dry, either at room temperature or in a dessicator at room temperature.

[image]

60 bp oligonucleotide, PCR primer #1-5′ GGCTATTGTT
GGTGGTGGTC 3′
60 bp oligonucleotide, PCR primer #2-5′ TCCAGCTTCA
GAAACCTGCT 3′
60 bp oligonucleotide-5′ GCTATTGTTGGTGGTGGTCCTGCTG
GTTTAGCCGTGGCTCAGCAGGTTTCTGAAGCTGGA 3′
(SEQ ID NOs:11-13, respectively)
70 bp oligonucleotide, PCR primer #1-5′ CAAACTCCAC
TGTGGTCTGC 3′
70 bp oligonucleotide, PCR primer #2-5′ AACCCAGTGG
CATCAAGAAC 3′
70 bp oligonucleotide-5′ AAACTCCACTGTGGTCTGCAGTGAC
GGTGTAAAGATTCAGGC TTCCGTGGTTCTTGATGCCACTGGGTT
(SEQ ID NOs:14-16, respectively)
80 bp oligonucleotide, PCR primer #1-5′ TGGTGTTCAT
GGATTGGAGA 3′
80 bp oligonucleotide, PCR primer #2-5′ GAACGTTGGG
ATCTTGCTGT 3′
80 bp oligonucleotide-5′ TGGTGTTCATGGATTGGAGAGACAA
ACATCTGGACTCATATC CTGAGCTGAAAGAACGGAACAGCAAGATCCCA
ACGTTC
(SEQ ID NOs:17-19, respectively)
90 bp oligonucleotide, PCR primer #1 5′ GGGGATCAAT
GTGAAGAGGA 3′
90 bp oligonucleotide, PCR primer #2 5′ CCACAACCCG
TTGAGGTAAG 3′
90 bp oligonucleotide-5′ GGGGATCAATGTGAAGAGGATTGAG
GAAGACGAGCGTTGTGTGATCCCGATGGGCGGTCCTTTACCAGTCTTACC
TCAACGGGTTGTGG
(SEQ ID NOs:20-22, respectively)

(SEQ ID NOs:20-22, respectively)

[image]

### EXAMPLE 2

This example describes an exemplary code using 50, 60, 70, 80, 90 and 100 base oligonucleotides in two sets (Sets #2 and #3).

Set #2
At3g59020 mRNA sequence
50 bp oligonucleotide, PCR primer #1-5′ GCACCCATTC
ACCGAGTAGT 3′
50 bp oligonucleotide, PCR primer #2-5′ ATGTTCAACA
GGTGGGGAAA 3′
50 bp oligonucleotide-5′ GCACCCATTCACCGAGTAGTCGAGG
AGACTTTTCCCCACCTGTTGAACAT 3′
(SEQ ID NOs:23-25, respectively)
60 bp oligonucleotide, PCR primer #1-5′ CAGTTTTTGC
TTTGCGTTCA 3′
60 bp oligonucleotide, PCR primer #2-5′ CTGGGCGGAT
TTCATCTAAA 3′
60 bp oligonucleotide-5′ CAGTTTTTGCTTTGCGTTCATTTAT
TGAAGCCTGCAAAGATTTAGATGAAATCCGCCCAG 3′
(SEQ ID NOs:26-28, respectively)
70 bp oligonucleotide, PCR primer #1-5′ TCAAGTGCCT
TCTGGTTGAA 3′
70 bp oligonucleotide, PCR primer #2-5′ AGTATGCCAA
GTGCCAAAGG 3′
70 bp oligonucleotide-5′ TCAAGTGCCTTCTGGTTGAAGTGGT
TGCAAATGCCTTTTACTACAATACCCTTTGGCACTTGGCATACT 3′
(SEQ ID NOs:29-31, respectively)
80 bp oligonucleotide, PCR primer #1-5′ TCGACACTGA
CAACGGTGAT 3′
80 bp oligonucleotide, PCR primer #2-5′ GGTACTGATG
GCACGGAGAC 3′
80 bp oligonucleotide-5′ TCGACACTGACAACGGTGATGATGA
AACTGATGATGCTGGTGCATTGGCTGCAGTGGGATGTCTCCGTGCCATCA
GTACC 3′
(SEQ ID NOs:32-34, respectively)
90 bp oligonucleotide, PCR primer #1-5′ CGAGTCTCGT
CGATTTCCTC 3′
90 bp oligonucleotide, PCR primer #2-5′ TTAAAGCGAG
GCTAGGCAGA 3′
90 bp oligonucleotide-5′ CGAGTCTCGTCGATTTCCTCCGGGA
GGAGACTTGAAATTCGTGACTTTCCGATTGTGAATTCCCCGATGGATCTG
CCTAGCCTCGCTTTAA 3′
(SEQ ID NOs:35-37, respectively)
100 bp oligonucleotide, PCR primer #1-5′ GTCTCCGTG
CCATCAGTACC 3′
100 bp oligonucleotide, PCR primer #2-5′ AGCATTTTC
CGCATTATTGG 3′
100 bp oligonucleotide-5′ GTCTCCGTGCCATCAGTACCATTC
TTGAATCTATCAGTAGTCTCCCTCATCTTTATGGTCAGATTGAACCACAG
TTACTGCCAATAATGCGGAAAATGCT 3′
(SEQ ID NOs:38-40, respectively)
Set #3
At5g18620 mRNA sequence
50 bp oligonucleotide, PCR primer #1-5′ TGTCTCTGAC
GACGAGGTTG 3′
50 bp oligonucleotide, PCR primer #2-5′ CGTCCTCTTC
AGCGTCATCT 3′
50 bp oligonucleotide-5′ TGTCTCTGACGACGAGGTTGTCCCC
GTAGAAGATGACGCTGAAGAGGACG 3′
(SEQ ID NOs:41-43, respectively)
60 bp oligonucleotide, PCR primer #1-5′ GGAGAACGCA
AACGTCTGTT 3′
60 bp oligonucleotide, PCR primer #2-5′ AAGGGTGATT
GCAGCATTTC 3′
60 bp oligonucleotide-5′ GGAGAACGCAAACGTCTGTTGAACA
TAGCAATGCATTGCGGAAATGCTGCAATCACCCT 3′
(SEQ ID NOs:44-46, respectively)
70 bp oligonucleotide, PCR primer #1-5′ AGGAACCCTC
GATTCGATCT 3′
70 bp oligonucleotide, PCR primer #2-5′ TCGAAGCTCT
AGCCATCGAC 3′
70 bp oligonucleotide-5′ AGGACCCTCGATTCGATCTCTCAGA
CGAAATCAGGATTCGTAGAGGCGCGTCGATGGCTAGAGCTTCGA 3′
(SEQ ID NOs:47-49, respectively)
80 bp oligonucleotide, PCR primer #1-5′ CCCTCGATTC
GATCTCTCAG 3′
80 bp oligonucleotide, PCR primer #2-5′ GAAGAAACTT
CCCGCTTCG 3′
80 bp oligonucleotide-5′ CCTCGATTCGATCTCTCAGACGAAA
TCAGGATTCGTAGAGGCGCGTCGATGGCTAGAGCTCGAAGCGGGAAGTTT
CTTC 3′
(SEQ ID NOs:50-52, respectively)
90 bp oligonucleotide, PCR primer #1-5′ CAGCAAACGT
GAGAAGGCTA 3′
90 bp oligonucleotide, PCR primer #2-5′ TGGAAGCATT
TTGGGAGTCT 3′
90 bp oligonucleotide-5′ CAGCAAACGTGAGAAGGCTAGACTC
AAAGAAATGCAGAAGATGAAGAAGCAGAAAATTCAGCAAATCTTAGACTC
CCAAAATGCTTCCA 3′
(SEQ ID NOs:53-55, respectively)
100 bp oligonucleotide, PCR primer #1-5′ GCCGATTTT
GTCCTGTCCT 3′
100 bp oligonucleotide, PCR primer #2-5′ ATGTCGAAT
TTCCCTGCAAC 3′
100 bp oligonucleotide-5′ GCCGATTTTGTCCTGTCCTGCGTG
CTGTGAAATTTCTCGGTAATCCCGAGGAAAGAAGACATATTCGTGAAGAA
CTGCTAGTTGCAGGGAAATTCGACAT 3′
(SEQ ID NOs:56-58, respectively)

Data Generated with Sets 2 and 3

With each set of primers being separated by 10 bases, a 6% polyacrylamide gel was employed (Invitrogen, Carlsbad). The PCR reaction conditions and the amount of oligonucleotide is as described above. The corresponding PCR primer concentration was reduced from 0.1 uM per reaction to 0.05 uM.

[image]

Enhancement of PCR with the Presence of the Bio-Tag

The addition of oligonucleotides to the matrix prior to the addition of blood enhances the amount of PCR product yield. The oligonucleotide code is applied to the matrix and allowed to dry completely prior to the addition of blood.

[image]

Beta Actin Primers

All reactions use the same primer #1: 5′ agcacagagcctcgccttt 3′

2 kb primer #2-5′ GGTGTGCACTTTTATTCAACTGG 3′
1.5 kb primer #2-5′ AGAGAAGTGGGGTGGCTTTT 3′
1.0 kb primer #2-5′ AGGGCAGTGATCTCCTTCTG 3′
0.5 kb primer #2-5′ AGAGGCGTACAGGGATAGCA 3′
(SEQ ID NOs:59-61, respectively)

### EXAMPLE 3

This example describes particular inherent properties of certain embodiments of the invention.

Inherent in the invention is the difficulty with which counterfeiters could identify and, therefore, reproduce the code. When using multiple (e.g., two or more) sets of oligonucleotides in which there is at least one oligonucleotide from the two sets having an identical length, it is impossible to reproduce the specific banding pattern created by the code without knowing the primers that specifically hybridize to the oligonucleotides. For example, although there are technologies that could provide the requisite sensitivity and resolution needed to visualize the bio-code on a gel without amplifying the oligonucleotides, this data would be worthless since there are at least two oligonucleotides having the same size in the code which could not be size-differentiated in one dimension. Furthermore, although random primed PCR could be attempted to clone and sequence the oligonucleotides comprising the code, this would simply generate a ladder up to the largest oligonucleotide present in the particular mixture, not the correct code pattern. When the oligonucleotides comprising the code are single strand, there is no practical way to clone single strand sequences into vectors to try and duplicate the combination of oligonucleotides comprising the code. Thus, in contrast to computer based encoding, electronic based authenticating markers, or watermarks which can eventually be duplicated with ever advancing computing capabilities, the code is not easily identified and, therefore, cannot be reproduced without knowing the sequences of the primers.

### EXAMPLE 4

This example describes various non-limiting specific applications of the bio-code.

Forensic Chain of Evidence Assurance: Forensic samples such as blood and body fluids or tissues that are collected at the scene of a crime or from a suspect using evidence collection kits based upon paper, or treated papers such as FTA (Whatman) or IsoCode (Schleicher and Schuell). A barcoded card is used to write down date, time, location, collector and other relevant information so that it stays with the collection card. When analysis of the sample on the collection card (e.g., nucleic acid) is desired, a 1 or 2 mm punch is taken from the portion of the collection card with the forensic sample, e.g., where the sample was collected. The nucleic acid is subsequently identified using commercially available human ID kits such as are provided by Promega and other commercial sources. These kits provide a buffer for washing the cellular debris and proteins from the nucleic acid purifying it for subsequent multiplex PCR for human identification.

A series of 25 different oligonucleotides chosen to avoid sequence commonality with the human genome are used to generate a unique bio-barcode similar to the exemplary illustration described herein. The unique code at a concentration set to provide a total of 5 ng/cm² is added to the card and allowed to dry. When the forensic sample is analyzed, for example, to ID the human based upon the DNA present, five additional PCR reactions are included to develop the bio-barcode. When the PCR reactions are fractionated via gel electrophoresis, the additional five lanes appear as barcode which is directly linked with the human ID information and with the sample on the original collection card. This method is advantageous because the means to develop the code are the same as that used to analyze the genetic material of the sample. Accordingly, the code directly links the ID of the individual to the information on the card used to collect the sample. Even though a punch might be initially mis-identified by a laboratory technician, all ambiguity is removed as soon as the bar-code of the punched section is developed. An additional feature is that a scan or digital image of the gel with both the nucleic acid sample and the bar-code will contain not only the identification information for the individual but also the direct link to the evidence, ensuring a rigid chain of custody to the location where the forensic sample was collected.

High Value Documents: Paper documents such as commercial paper, bonds, stocks, money, etc. can be ensured to be authentic by implanting upon the paper and valid copies, a unique combination of oligonucleotides providing a barcode. If the validity of the document is in question, a sample of the paper is taken and the code developed, for example, via PCR amplification and subsequent gel electrophoresis. If the barcode is absent or does not match the expected code, then the item is counterfeit. Similarly, by the attachment of a small swatch of paper or fabric to any high value item, authenticity of the item can be ensured.

Again, the use of 25 primer pairs that specifically hybridize to 25 oligonucleotides in a binary (present or not present) code can be use to uniquely identify over 34 million different documents. By using 30 oligonucleotides and six lanes of 5 primer pairs each, the system can be used to uniquely identify over one billion different documents. Cost per document can be as low as a few cents or less if the code material is placed in a specific location on the document such as part of the letterhead or a designated area of the print information on the document. A wax or other seal (organic or inorganic) could also be placed over the code material to protect against possible loss or degradation.

Sample Storage/Archiving: In an automated sample store (i.e., archive), study assembly consists of selecting multiple samples from the archive and assembling them into a daughter plate (typically a lab microplate consists of 100 to 1000 wells, each capable of containing a distinct sample). Clinical samples of this type are typically valued at about $100 each, so mistakes in sample assembly or a mishap during or after sample retrieval resulting in the samples being scrambled would be extremely costly. Although some of this risk can be avoided through careful package and process design (i.e., sample storage, retrieval and tracking), a code for each sample when the sample is introduced into the archive so that the sample can be distinguished from others and traced back to their original source provides additional protection.

One can code every sample that enters the sample store. However, it is not necessary to code every sampler. For example, samples can be coded upon retrieval from the store, which is more economical since fewer codes are required and because the coding expense is incurred only for those samples that leave the archive rather than for every sample that enters the archive. In any event, the oligonucleotide code can be added to or mixed with every sample introduced into the store or only those samples that leave the store.

1. A composition comprising two or more oligonucleotides and a sample, said oligonucleotides denoted a first oligonucleotide set, said first oligonucleotide set comprising oligonucleotides incapable of specifically hybridizing to said sample, said oligonucleotides having a length from about 8 nucleotides to 50 Kb, said first oligonucleotide set comprising oligonucleotides each having a physical or chemical difference from the other oligonucleotides comprising said first oligonucleotide set, said first oligonucleotide set comprising one or more oligonucleotides having a different sequence therein capable of specifically hybridizing to a unique primer pair denoted a first primer set.

2. The composition of claim 1, wherein the difference comprises oligonucleotide length.

3. The composition of claim 1, wherein the two oligonucleotides are denoted A through B and the unique combination comprises A with or without B; or B with or without A.

4. The composition of claim 1, wherein three oligonucleotides are denoted A through C and the unique combination comprises A with or without B or C; B with or without A or C; or C with or without A or B.

5. The composition of claim 1, wherein four oligonucleotides are denoted A through D and the unique combination comprises A with or without B or C or D; B with or without A or C or D; C with or without A or B or D; or D with or without A or B or C.

6. The composition of claim 1, wherein five oligonucleotides are denoted A through E and the unique combination comprises A with or without B or C or D or E; B with or without A or C or D or E; C with or without A or B or D or E; D with or without A or B or C or E; or E with or without A or B or C or D.

7. The composition of claim 1, wherein six oligonucleotides are denoted A through F and the unique combination comprises A with or without B or C or D or E or F; B with or without A or C or D or E or F; C with or without A or B or D or E or F; D with or without A or B or C or E or F; E with or without A or B or C or D or F; or F with or without A or B or C or D or E.

8. The composition of claim 1, wherein seven oligonucleotides are denoted A through G and the unique combination comprises A with or without B or C or D or E or F or G; B with or without A or C or D or E or F or G; C with or without A or B or D or E or F or G; D with or without A or B or C or E or F or G; E with or without A or B or C or D or F or G; F with or without A or B or C or D or E or G; or G with or without A or B or C or D or E or F.

9. The composition of claim 1, comprising a unique combination of two to five, five to ten, 10 to 15, 15 to 20, 20 to 25, 25 to 30, 30 to 40, 40 to 50, or more oligonucleotides.

10. The composition of claim 1, wherein the oligonucleotides have a length from about 10 to 5000 base pairs.

11.-129. (canceled)
